(19) United States
(12) Patent Application Publication
(10) Pub. No.: US 2024/0067932 A1
(43) Pub. Date: Feb. 29, 2024

(54) METHODS AND COMPOSITIONS FOR GENERATING HEMOGENIC ENDOTHELIAL CELLS FROM PLURIPOTENT STEM CELLS

(71) Applicant: Trailhead Biosystems Inc., Cleveland, OH (US)

(72) Inventors: Angelica M. GOMES UELTSCHY, Shaker Heights, OH (US); Michael G. ELIAS, Cleveland, OH (US)

(21) Appl. No.: 18/238,139

(22) Filed: Aug. 25, 2023

Related U.S. Application Data
(60) Provisional application No. 63/400,944, filed on Aug. 25, 2022.

(51) Int. Cl. C12N 5/071

(52) U.S. Cl. CPC C12N 5/069; C12N 2500/38; C12N 2501/115; C12N 2501/155; C12N 2501/165; C12N 2501/41; C12N 2501/415; C12N 2501/70; C12N 2501/998; C12N 2506/45

(57) ABSTRACT

Methods for generating CD31+CD34+CD143+CD309+GATA2+FLI1+RUNX1+vWF+hemogenic endothelial cells (HECs) are provided using chemically-defined culture media that allow for generating HECs from early mesoderm progenitors in as little as three days and from pluripotent stem cells in as little as five days. Culture media, isolated cell populations and kits are also provided.

### RELATED APPLICATION

This application claims priority to U.S. Provisional Application No. 63/400,944, filed Aug. 25, 2022, the entire contents of which is hereby incorporated by reference.

### BACKGROUND OF THE INVENTION

Vascular endothelial cells (ECs) are the main type of cells lining the blood vessels throughout the body. During embryonic development, a specialized subset of vascular endothelial cells, termed hemogenic endothelial cells, acquires the potential to form blood cells, giving rise to hematopoietic stem and progenitor cells (HSPCs). Hemogenic endothelial cells represent a small (1-3% of endothelial cells in distinct tissues), transient population of specialized cells that arise to initiate definitive hematopoiesis (Gritz and Hirschi (2016) Cell. Mol. Life Sci. 73:1547-1567). HSPCs function to generate all blood cells in the body, both throughout embryonic development and in adult life. For an overview of the regulation of hemogenic endothelial cell development and function, see e.g., Wu and Hirschi (2021) Ann. Rev. Physiol. 83:17-37 and Lange et al. (2021) Cell. Mol. Life Sci. 78:4143-4160.

The earliest methodologies for generating endothelial cells from pluripotent stem cells devised three different approaches (as reviewed in, for example, Wilson et al. (2014) Stem Cells 32:3037-3045; Yoder (2015) Curr. Opin. Hematol. 22:252-257; Williams and Wu (2019) Arter. Thromb. Vasc. Biol. 39:1317-1329). In the first approach, embryonic stem cells (ESCs) or induced pluripotent stem cells (iPSCs) are grown under conditions that induce self-aggregation of the cells into embryoid bodies (EBs) and then a variety of growth factors are used to promote endothelial-lineage differentiation with the EBs. In the second approach, differentiating ESCs or iPSCs are co-cultured with a feeder layer, such as stromal cells (e.g., murine calvarial mesenchymal OP9 cells or bone marrow stromal cells), to promote differentiation along the endothelial lineage. In the third approach, ESCs or iPSCs are grown in two-dimensional culture on plates coated with a protein substrate, such as Matrigel, gelatin, fibronectin or similar protein, under specific conditions with added growth factors or small molecules.

Activation of canonical Wnt signaling has been reported to be involved in promoting hemogenic endothelial cell differentiation from pluripotent stem cells. Transient treatment of iPSCs with a GSK-3β inhibitor (to thereby activate the Wnt signaling pathway) was shown to trigger activation of the CDX/HOX pathway, leading to hemogenic posterior mesoderm differentiation of the iPSCs (Kitajima et al. (2016) Exp. Hematol. 44:68-74). A serum-free system using culture with a GSK-3β inhibitor has been described for hemogenic endothelial cell differentiation from human pluripotent stem cells (Galat et al. (2017) Stem Cell Res. Therap. 8:67). Subsequent protocols for generating hemogenic endothelial cells have combined Wnt pathway activation with treatment with growth factors such as VEGF, FGF2 and BMP4 (see e.g., Bruveris et al. (2020) Development 147:dev193037).

Accordingly, while some progress has been made, there remains a need for efficient and robust methods and compositions for generating endothelial cells from pluripotent stem cells in culture.

### SUMMARY OF THE INVENTION

This disclosure provides methods of generating hemogenic endothelial cells (HECs) using chemically-defined culture media that allows for robust generation of CD31+CD34+D143+CD309+GATA2+FLI1+RUNX1+vWF+HECs from pluripotent stem cells in as little as five days of culture. The culture media comprises small molecule agents that either agonize or antagonize particular signaling pathways such that differentiation along the endothelial cell lineage is promoted. Further culture of the resultant hemogenic endothelial cells under blood lineage-specific cell culture conditions leads to differentiation into both short term and long-term hematopoietic stem cells. Additionally, the use of small molecule agents in the culture media allows for precise control of the culture components.

The methods and compositions of the disclosure utilize a unique cell culture media that allows for generation of hemogenic endothelial cells from early mesoderm progenitor cells in as little as three days. This unique cell culture media includes certain components previously used for endothelial cell generation, such as a VEGFR agonist and an FGFR agonist, but also includes novel components and combinations that allow for robust hemogenic endothelial cell generation. Early mesoderm progenitor cells can be obtained by culturing pluripotent stem cells under culture conditions that generate the early mesoderm progenitors, as described herein. Thus, in certain embodiments, the methods comprise a single stage culture protocol, starting with early mesoderm progenitors and resulting in CD31+CD34+CD143+CD309+GATA2+FLI1+RUNX1+vWF+hemogenic endothelial cells in three days. In other embodiments, the methods comprise a two-stage culture protocol, starting with pluripotent stem cells being differentiated for two days into early mesoderm progenitors in stage 1 and then further differentiating the early mesoderm progenitors for three days into CD31+CD34+CD143+CD309+GATA2+FLI1+RUNX1+vWF+hemogenic endothelial cells as stage 2, resulting in a two-stage, five day protocol.

Accordingly, in one aspect, the disclosure pertains to a method of generating human CD31+CD34+CD143+CD309+GATA2+FLI1+RUNX1+vWF+HECs comprising:
culturing human early mesoderm progenitor cells in a culture media comprising a VEGFR agonist, an FGFR agonist, a sonic hedgehog (SHH) agonist, an adenylyl cyclase activator, an actin-binding protein, a BMP pathway agonist, a Wnt pathway antagonist, and a retinoic acid (RA) receptor agonist to generate human CD31+CD34+CD143+CD309+GATA2+FLI1+RUNX1+vWF+HECs.

In an embodiment, the early mesoderm progenitor cells are cultured for three days in the culture media to obtain human CD31+CD34+CD143+CD309+GATA2+FLI1+RUNX1+vWF+HECs.

In an embodiment, the early mesoderm progenitor cells are obtained by culturing human pluripotent stem cells in a media comprising a Wnt pathway agonist for two days.

In another aspect, the disclosure pertains to a two stage method of generating human CD31+CD34+CD143+CD309+GATA2+FLI1+RUNX1+vWF+hemogenic endothelial cells (ECs), the method comprising:
(a) culturing human pluripotent stem cells in a culture media comprising a Wnt pathway agonist on day 0-day 2 to generate early mesoderm progenitor cells; and
(b) culturing the early mesoderm progenitor cells in a culture media comprising a VEGFR agonist, an FGFR agonist, a sonic hedgehog (SHH) agonist, an adenylyl cyclase activator, an actin-binding protein, a BMP pathway agonist, a Wnt pathway antagonist, and a retinoic acid (RA) receptor agonist on day 2-day 5 to generate CD31+CD34+CD143+CD309+GATA2+FLI1+RUNX1+vWF+HECs.

In an embodiment, the Wnt pathway agonist used in the culture media for generating early mesoderm progenitors is a GSK-3β inhibitor. In an embodiment, the GSK-3β inhibitor is CHIR99021. In an embodiment, CHIR99021 is present in the culture at a concentration in a range of 3.0-9.0 μM. In an embodiment, CHIR99021 is present in the culture at a concentration of 6.0 μM.

In an embodiment, the VEGFR agonist is VEGF. In an embodiment, VEGF is present in the culture at a concentration in a range of 25-75 ng/ml. In an embodiment, VEGF is present in the culture at a concentration of 50 ng/ml.

In an embodiment, the FGFR agonist is FGF2 or SUN11602. In an embodiment, the FGFR agonist is present in the culture at a concentration in a range of 1-20 ng/ml. In an embodiment, the FGFR agonist is FGF2, which is present in the culture at a concentration in a range of 1-20 ng/ml. In an embodiment, the FGFR agonist is FGF2, which is present in the culture at a concentration of 10 ng/ml.

In an embodiment, the sonic hedgehog (SHH) agonist is selected from the group consisting of Purmorphamine, SSH, GSA 10, SAG, and combinations thereof. In an embodiment, the SHH agonist is present in the culture at a concentration in a range of 100-1000 nM. In an embodiment, the SHH agonist is Purmorphamine, which is present in the culture at a concentration in a range of 400-600 nM. In an embodiment, the SHH agonist is Purmorphamine, which is present in the culture at a concentration of 500 nM.

In an embodiment, the adenylyl cyclase activator is selected from the group consisting of Forskolin, NKH 477, PACAP 1-27, PACAP 1-38, Adenosine, Carbacyclin, Dopamine, Endothelin 1, Endothelin 1, L-(−)-Epinephrine-(+)-bitartrate, Glucagon, Isoproterenol HCl, (±)-Octopamine HCl, Parathyroid Hormone 1-34, Prostaglandin D₂, Prostaglandin E₁, Prostaglandin E₂, Prostaglandin I₂, [Arg⁸]-Vasopressin, [Lys⁸]-Vasopressin, and combinations thereof. In an embodiment, the adenylyl cyclase activator is present in the culture at a concentration in a range of 0.1-10 μM. In an embodiment, the adenylyl cyclase activator is Forskolin, which is present in the culture at a concentration in a range of 0.75-2.5 μM. In an embodiment, the adenylyl cyclase activator is Forskolin, which is present in the culture at a concentration of 1.0 μM.

In an embodiment, the actin binding protein is selected from the group consisting of thymosyin-β4, HMRef, α-actinin, β-spectrin, dystrophin, utrophin, fimbrin, and combinations thereof. In an embodiment, the actin binding protein is present in the culture at a concentration in a range of 0.1-10 μg/ml. In an embodiment, the actin binding protein is thymosyin-β4, which is present in the culture at a concentration in a range of 0.75-2.5 μg/ml. In an embodiment, the actin binding protein is thymosyin-β4, which is present in the culture at a concentration of 1 μg/ml.

In an embodiment, the BMP pathway agonist is selected from the group consisting of BMP4, BMP2, BMP6, BMP7, GDF6, and combinations thereof. In an embodiment, the BMP pathway agonist is present in the culture at a concentration in a range of 5-50 ng/ml. In an embodiment, the BMP pathway agonist is BMP4, which is present in the culture at a concentration in a range of 15-30 ng/ml. In an embodiment, the BMP pathway agonist is BMP4, which is present in the culture at a concentration of 20 ng/ml.

In an embodiment, the Wnt pathway antagonist is selected from the group consisting of XAV939, ICG-001 (Foscenvivint), Capmatinib (INCB28060), endo-IWR-1, IWP-2, IWP-4, MSAB, CCT251545, KY02111, NCB-0846, FH535, LF3, WIKI4, Triptonide, KYA1797K, JW55, JW 67, JW74, Cardionogen 1, NLS-StAx-h, TAK715, PNU 74654, iCRT3, iCRT14, WIF-1, DKK1, Isoquercitrin, Lanatoside C, Gigantol, RCM-1, WIKI4, IQ-1, Adavivant, PRI-724, Tegatrabetan, or combinations thereof. In an embodiment, the Wnt pathway antagonist is present in the culture at a concentration in a range of 10-500 nM. In an embodiment, the Wnt pathway antagonist is XAV939, which is present in the culture at a concentration in a range of 50-150 nM. In an embodiment, the Wnt pathway antagonist is XAV939, which is present in the culture at a concentration of 100 nM.

In an embodiment, the retinoic acid (RA) pathway agonist is selected from the group consisting of retinoic acid (RA), TTNPB, AM 580, CD 1530, CD 2314, CD 437, Ch 55, BMS 753, BMS 961, Tazarotene, Tamibarotene, Isotretinoin, Tretinoin, AC 261066, AC 55649, Sr11237, adapalene, EC23, 9-cis retinoic acid, 13-cis retinoic acid, 4-oxo retinoic acid, and All-trans Retinoic Acid (ATRA), AY 9944 dihydrochloride, Ciliobrevin A, Cyclopamine, or combinations thereof. In an embodiment, the RA pathway agonist is present in the culture at a concentration in a range of 100-1000 nM. In an embodiment, the RA pathway agonist is TTNPB, which is present in the culture at a concentration in a range of 400-600 nM. In an embodiment, the RA pathway agonist is TTNPB, which is present in the culture at a concentration of 500 nM.

In an embodiment, the pluripotent stem cells are embryonic stem cells. In an embodiment, the pluripotent stem cells are induced pluripotent stem cells.

In another aspect, the disclosure pertains to a culture media for generating HECs comprising a VEGFR agonist, an FGFR agonist, a sonic hedgehog (SHH) agonist, an adenylyl cyclase activator, an actin-binding protein, a BMP pathway agonist, a Wnt pathway antagonist, and a retinoic acid (RA) receptor agonist. In an embodiment, the VEGFR agonist is VEGF, the FGFR agonist is FGF2, the SHH agonist is Purmorphamine, the adenylyl cyclase activator is Forskolin, the actin binding protein is thymosin-β4, the BMP pathway agonist is BMP4, the Wnt pathway antagonist is XAV939 and the RA receptor agonist is retinoic acid. In an embodiment, VEGF is at a concentration of 50 ng/ml, FGF2 is at a concentration of 10 ng/ml, Purmorphamine is at a concentration of 500 nM, Forskolin is at a concentration of 1 μM, thymosin-β4 is at a concentration of 1 μg/ml, BMP4 is at a concentration of 20 ng/ml, XAV939 is at a concentration of 100 nM and retinoic acid is at a concentration of 500 nM.

In yet another aspect, the disclosure pertains to an isolated cell culture of human hemogenic endothelial cells, the culture comprising: human CD31+CD34+CD143+CD309+GATA2+FLI1+RUNX1+vWF+HECs cultured in a culture media comprising a VEGFR agonist, an FGFR agonist, a sonic hedgehog (SHH) agonist, an adenylyl cyclase activator, an actin-binding protein, a BMP pathway agonist, a Wnt pathway antagonist, and a retinoic acid (RA) receptor agonist.

Other features and advantages of the invention will be apparent from the following detailed description and claims.

### BRIEF DESCRIPTION OF THE DRAWINGS

FIG. 1 is a schematic diagram of a representative culture protocol for generating hemogenic endothelial cells from early mesoderm progenitors in three days, as well as the two-day pre-treatment to generate the early mesoderm progenitors from iPSCs.

FIG. 2 shows the results from an HD-DoE model of an 8-factor experiment optimized for maximum expression of GATA2. The upper section of the model shows the prediction of expression level of pre-selected 52 genes when optimized for GATA2. The lower section of the model shows the effectors that were tested in this model and their contribution to maximum expression of GATA2. The value column refers to required concentration of each effector to mimic the model.

FIG. 3 shows the results from an HD-DoE model of an 8-factor experiment optimized for maximum expression of GATA2. Upper and lower sections are as described in FIG. 2.

FIG. 4 shows the results from an HD-DoE model of an 8-factor experiment optimized for maximum expression of GATA2. Upper and lower sections are as described in FIG. 2.

FIG. 5 shows the results from an HD-DoE model of an 8-factor experiment optimized for maximum expression of GATA2. Upper and lower sections are as described in FIG. 2.

FIG. 6 shows the results from an HD-DoE model of an 8-factor experiment optimized for maximum expression of GATA2. Upper and lower sections are as described in FIG. 2.

FIGS. 7A-7B show the dynamic profile analysis of expression levels of GATA2, PECAM1, CD309 (KDR), CD34, FLI1, TAL1, and CD44 relative to the concentration of 3 validated effectors. The impact of VEGF, Forskolin, and FGF2 on expression of genes above and their factor contributions are shown by slope of the plots for each effector.

FIGS. 8A-8B show the dynamic profile analysis of expression levels of GATA2, PECAM1, CD309 (KDR), CD34, FLI1, TAL1, and CD44 relative to the concentration of 3 validated effectors. The impact of VEGF, Thymosin-β4, and FGF2 on expression of genes above and their factor contributions are shown by slope of the plots for each effector.

FIGS. 9A-9B show the dynamic profile analysis of expression levels of GATA2, PECAM1, CD309 (KDR), CD34, FLI1, TAL1, and CD44 relative to the concentration of 3 validated effectors. The impact of Purmorphamine, VEGF, and B27 on expression of genes above and their factor contributions are shown by slope of the plots for each effector.

FIGS. 10A-10B show the dynamic profile analysis of expression levels of GATA2, PECAM1, CD309 (KDR), CD34, FLI1, TAL1, and CD44 relative to the concentration of 4 validated effectors. The impact of XAV939, VEGF, FGF2, and L-ascorbic acid on expression of genes above and their factor contributions are shown by slope of the plots for each effector.

FIGS. 11A-11B show the dynamic profile analysis of expression levels of GATA2, PECAM1, CD309 (KDR), CD34, FLI1, TAL1, and CD44 relative to the concentration of 3 validated effectors. The impact of Retinoic Acid, BMP4, and VEGF on expression of genes above and their factor contributions are shown by slope of the plots for each effector.

FIGS. 12A-12B show results of flow cytometry staining of iPSC-derived HECs at the end of stage 2. FIG. 12A shows cells that were stained with CD31, CD34, CD61, CD143, and CD309 (KDR). At this stage, cells were positive for all the markers expected for HECs. n=5 for CD31, CD34, CD61, and KDR and n=2 for CD143. FIG. 12B portrays the increase in CD31, CD34, and CD61 with the culture media described herein containing small molecules vs the known endothelial cell differentiation proteins VEGF and FGF2 alone.

FIG. 13A shows a representative plot of the organization of endothelial (blue) and hemogenic endothelial (red) lineage determinant genes set as induced by factors combinations on mesoderm committed cells. Prior addition of factors, cells were exposed to stage 1 media for 2 days. The plot was generated by HDB-unsupervised clustering algorithm. Conditions have been identified for expression of genes enriched in hemogenic endothelial cells such as, GATA2, TAL1, and ETV2, as well as for genes expressed by endothelial cells such as PECAM1 and ERG.

FIG. 13B are photographs showing RUNX1 positive HECs differentiated from iPSC using the stage 2 recipe described herein and RUNX1negative endothelial cells using a different stage 2 recipe optimized for endothelial cell differentiation.

FIG. 14 shows photographs of fluorescence images of iPSC-derived hemogenic endothelial cells at the end of stage 2. Cells were stained with hemogenic endothelial cell biomarkers including CD31, FLI1, CD144, CD309 (KDR), vWF, RUNX1, GATA2, and SOX17. At this stage, cells were positive for all the markers expected for hemogenic endothelial cells.

FIG. 15 shows results of flow cytometry staining of iPSC-derived HECs at the end of stage 2 from 2 iPSC cell lines (iX Cells CR0000001 and REPROCELL 771-3G). Cells were stained with HEC markers RUNX1, CD34, CD43, and CD31. Both CR0000001 and 771-3G cell lines were positive at expected levels of expression for all the markers. Both cell lines showed similar expression levels of the HEC markers.

FIG. 16 shows the results of bulk RNA-seq used to characterize the iPSC-derived HECs. iPSCs were differentiated to lateral plate mesoderm as previously described (stage 1). Then, mesodermal cells were treated with stage 2 media for 3 days. The heat map shows the Z-score of transcripts per kilobase million (TPM) from the bulk RNA-seq characterization. At the end of stage 2, RNA-seq demonstrated upregulation of stage 2 HEC genes and downregulation stage 0 and stage 1 pluripotency, primitive streak and mesoderm genes. TPM values above the mean are shown in red and below the mean are shown in blue.

### DETAILED DESCRIPTION OF THE INVENTION

Described herein are methodologies and compositions that allow for the robust generation of CD31+CD34+CD143+CD309+GATA2+FLI1+RUNX1+vWF+hemogenic endothelial cells from iPSCs in only five days under chemically-defined culture conditions using a small molecule based approach. A High-Dimensional Design of Experiments (HD-DoE) approach was used to simultaneously test multiple process inputs (e.g., small molecule agonists or antagonists) on output responses, such as gene expression. These experiments allowed for the identification of chemically-defined culture media, comprising agonists and/or antagonists of particular signaling pathways, that is sufficient to generate HECs from iPSCs in a very short amount of time. The optimized culture media was further validated by a factor criticality analysis, which examined the effects of eliminating individual agonist or antagonist agents. Flow cytometry and immunocytochemistry analysis were used to further confirm the phenotype of the cells generated by the differentiation protocol.

Various aspects of the invention are described in further detail in the following subsections.

### I. Cells

The starting cells used in the cultures of the disclosure typically are human pluripotent stem cells, which are used to generate early mesoderm progenitors that are then used to obtain hemogenic endothelial cells. As used herein, the term “human pluripotent stem cell” (abbreviated as hPSC) refers to a human stem cell that has the capacity to differentiate into a variety of different cell types. The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, for example, using a nude mouse and teratomas formation assay. Pluripotency can also evidenced by the expression of embryonic stem (ES) cell markers, although the preferred test for pluripotency is the demonstration of the capacity to differentiate into cells of each of the three germ layers.

Human pluripotent stem cells include, for example, induced pluripotent stem cells (iPSCs) and human embryonic stem cells, such as ES cell lines. Non-limiting examples of induced iPSCs include 19-11-1, 19-9-7 or 6-9-9 cells (e.g., as described in Yu, J. et al. (2009) Science 324:797-801). Non-limiting examples of human embryonic stem cell lines include ES03 cells (WiCell Research Institute) and H9 cells (Thomson, J. A. et al. (1998) Science 282:1145-1147). hPSCs express cellular markers that can be used to identify cells as being PSCs. Non-limiting examples of pluripotent stem cell markers include TRA-1-60, TRA-1-81, TRA-2-54, SSEA1, SSEA3, SSEA4, CD9, CD24, OCT3, OCT4, NANOG and/or SOX2. Since the methods of generating HECs of the disclosure are used to differentiate (maturate) the starting pluripotent stem cell population, in various embodiments HEC populations generated by the methods of the disclosure lack expression of one or more stem cell markers, such as one or more stem cell markers selected from the group consisting of TRA-1-60, TRA-1-81, TRA-2-54, SSEA1, SSEA3, SSEA4, CD9, CD24, OCT3, OCT4, NANOG and/or SOX2.

The pluripotent stem cells are subjected to culture conditions, as described herein, that induce cellular differentiation. As used herein, the term “differentiation” refers to the development of a cell from a more primitive stage towards a more mature (i.e., less primitive) cell, typically exhibiting phenotypic features of commitment to a particular cellular lineage. For generation of hemogenic endothelial cells from the pluripotent stem cells, the stem cells are first differentiated to mesoderm commitment.

As used herein, a “early mesoderm progenitor” refers to a cell that is more differentiated than a pluripotent stem cell and which is committed to the mesodermal lineage. As described herein, an early mesoderm progenitor can be obtained from a PSC by culture with an agent that activates Wnt signaling, such as a GSK-3β inhibitor (e.g., CHIR99021 for two days).

In embodiments, cells can be identified and characterized based on expression of one or more biomarkers, such as particular biomarkers of early mesoderm progenitors or of differentiated endothelial cells. Non-limiting examples of biomarkers whose expression can be assessed in the characterization of cells of interest include Brachyury (T) and KDR as biomarkers for early mesoderm progenitors and CD31, CD34, CD143, CD309, GATA2, FLI1, RUNX1, and vWF as biomarkers for differentiated hemogenic endothelial cells.

GATA2 is a transcription factor that has been identified as a master regulator of hematopoiesis and promotes the generation of HECs (Castano, J. et al. (2019) Stem Cell Reports 13:515-529). RUNX1 is a transcription factor that governs the emergence of definitive HECs and is widely recognized as a key marker for them (Ling, M. et al. (2014) Blood 11:e11-e20). Both master hemogenic regulators, GATA2 and RUNX1, have shown a parallel expression increase in iPSC derived HECs (Castano, J. et al. (2019) Stem Cell Reports 13:515-529). SOX17 is a transcription factor that has been identified as master regulator of the arterial program in the hemogenic endothelium and is required for the specification of hemogenic endothelial cells (Jung et al. (2021) Cell Rep. 34:108758). CD143 (ACE), has been shown to mark early hematopoietic stem cells (Fadlullah, M. Z. H., et al. (2022) Blood 139:343-346; Jokubaitis, V. et al. (2008) Blood 111:4055-4063). CD44 has been demonstrated to be a marker for HECs and a regulator for endothelial to hematopoietic transition (Oatley, M. et al. (2020) Nature Communications 11:586). Additionally, TAL1 is a transcription factor which is essential for maintaining HSPC multipotency and can be detected in primitive HSPCs, such as HECs (Real, P. et al. (2012) Molecular Therapy 20:1443-1453).

### II. Culture Media Components

The method of the disclosure for generating hemogenic endothelial cells from pluripotent stem cells comprise culturing early mesoderm progenitors in a culture media comprising specific agonist and/or antagonists of cellular receptors and/or signaling pathways to generate differentiated HECs. Moreover, the early mesoderm progenitors first can be obtained from pluripotent stem cells (e.g., ESCs or iPSCs) by culture of the stem cells under defined culture conditions as described herein.

A culture media comprising a VEGFR agonist, an FGFR agonist, a sonic hedgehog (SHH) agonist, an adenylyl cyclase activator, an actin-binding protein, a BMP pathway agonist, a Wnt pathway antagonist, and a retinoic acid (RA) receptor agonist is sufficient to generate CD31+CD34+CD143+CD309+GATA2+FLI1+RUNX1+vWF+hemogenic endothelial cells from early mesoderm progenitor cells in as little three days of culture. The early mesoderm progenitors can be obtained from PSCs by culture of the PSCs with a Wnt pathway agonist for two days, leading to an overall five day protocol to obtain differentiated HECs from PSCs under defined culture conditions.

As used herein, an “agonist” of a cellular receptor or signaling pathway is intended to refer to an agent that stimulates (upregulates) the cellular receptor or signaling pathway. Stimulation of the cellular signaling pathway can be initiated extracellularly, for example by use of an agonist that activates a cell surface receptor involved in the signaling pathway (e.g., the agonist can be a receptor ligand). Additionally or alternatively, stimulation of cellular signaling can be initiated intracellularly, for example by use of a small molecule agonist that interacts intracellularly with a component(s) of the signaling pathway.

As used herein, an “antagonist” of a cellular signaling pathway is intended to refer to an agent that inhibits (downregulates) the cellular signaling pathway. Inhibition of the cellular signaling pathway can be initiated extracellularly, for example by use of an antagonist that blocks a cell surface receptor involved in the signaling pathway. Additionally or alternatively, inhibition of cellular signaling can be initiated intracellularly, for example by use of a small molecule antagonist that interacts intracellularly with a component(s) of the signaling pathway.

Agonists and antagonists used in the chemically-defined media and methods of the disclosure are known in the art and commercially available. They are used in the culture media at a concentration effective to achieve the desired outcome, e.g., generation of cells of interest (such as early mesoderm progenitors or differentiated endothelial cells) expressing markers of interest. Non-limiting examples of suitable agonist and antagonists agents, and effective concentration ranges, are described further below.

Agonists of the VEGFR pathway include agents, molecules, compounds, or substances capable of stimulating (upregulating) the vascular endothelial growth factor receptor signaling pathway, which biologically is activated by binding of VEGF to VEGFR. In an embodiment, the VEGFR agonist is VEGF or an analog thereof that stimulates signaling through VEGR. In an embodiment, the VEGFR agonist is VEGF (e.g., recombinant human VEGF). In an embodiment, the VEGFR agonist is VEGF, which is present in the culture media at a concentration within a range of 10-100 ng/ml, 25-75 ng/ml, 40-60 ng/ml, 45-55 ng/ml or at a concentration of 50 ng/ml.

Agonists of the FGFR pathway include agents, molecules, compounds, or substances capable of stimulating (upregulating) a fibroblast growth factor receptor signaling pathway, which biologically is activated by binding of an FGF to an FGFR. In an embodiment, the FGFR agonist is FGF2, SUN11602, or combinations thereof. In an embodiment, the FGFR pathway agonist is present in the culture media at a concentration within a range of 1-20 ng/ml, 5-15 ng/ml, 7.5-12.5 ng/ml, 9-11 ng/ml or at a concentration of 10 ng/ml. In an embodiment, the FGFR agonist is FGF2 (e.g., recombinant human FGF2). In an embodiment, the FGFR agonist is FGF2 which is present in the culture media at a concentration within a range of 1-20 ng/ml, 5-15 ng/ml, 7.5-12.5 ng/ml, 9-11 ng/ml or at a concentration of 10 ng/ml.

Agonists of the SHH (sonic hedgehog) pathway include agents, molecules, compounds, or substances capable of stimulating (activating) signaling through the SHH pathway, which biologically involves binding of SHH to the Patched-1 (PTCH1) receptor and transduction through the Smoothened (SMO) transmembrane protein. In one embodiment, the SHH pathway agonist is selected from the group consisting of Purmorphamine, SSH, GSA 10, SAG, and combinations thereof. In one embodiment, the SHH pathway agonist is present in the culture media at a concentration within a range of 100-1000 nM, 200-800 nM, 250-750 nM or 450-550 nM, or at a concentration of 500 nM. In one embodiment, the SHH pathway agonist is Purmorphamine. In one embodiment, the SHH pathway agonist is Purmorphamine, which is present in the culture media at a concentration of 100-1000 nM, 200-800 nM, 250-750 nM or 500-600 nM. In one embodiment, the SHH pathway agonist is Purmorphamine, which is present in the culture media at a concentration of 500 nM.

Activators of adenylyl cyclase include agents, molecules, compounds, or substances capable of stimulating (upregulating) the activity of an adenylyl cyclase enzyme (also known in the art as adenyl cyclase and adenylate cyclase), which catalyzes the conversion of ATP to cAMP and pyrophosphate. In one embodiment, the adenylyl cyclase activator is selected from the group consisting of Forskolin, NKH 477, PACAP 1-27, PACAP 1-38, Adenosine, Carbacyclin, Dopamine, Endothelin 1, Endothelin 1, L-(−)-Epinephrine-(+)-bitartrate, Glucagon, Isoproterenol HCl, (±)-Octopamine HCl, Parathyroid Hormone 1-34, Prostaglandin D₂, Prostaglandin E₁, Prostaglandin E₂, Prostaglandin 12, [Arg⁸]-Vasopressin, [Lys⁸]-Vasopressin, and combinations thereof. In one embodiment, the adenylyl cyclase activator is present in the culture media at a concentration within a range of 0.1-10 μM, 0.5-5 μM, 0.75-2.5 μM or 0.9-1.1 μM, or at a concentration of 1 μM. In one embodiment, the adenylyl cyclase activator is Forskolin. In one embodiment, the adenylyl cyclase activator is Forskolin, which is present in the culture media at a concentration within a range of 0.1-10 μM, 0.5-5 μM, 0.75-2.5 μM or 0.9-1.1 μM. In one embodiment, the adenylyl cyclase activator is Forskolin, which is present in the culture media at a concentration of 1.0 μM.

Actin binding proteins (also known as ABPs) are proteins that bind to actin monomers, actin polymers or both. An extensive number of ABPs are known in the art, non-limiting examples of which include thymosyin-β4, HMRef, α-aetinin, β-spectrin, dystrophin, utrophin and fimbrin. In one embodiment, the ABP is a thymosin. In one embodiment, the thymosin is thymosyinβ4 or thymosyin-α1. In one embodiment, the actin binding protein is present in the culture media at a concentration within a range of 0.1-10 μg/ml, 0.5-5 μg/ml, 0.75-2.5 μg/ml or 0.9-1.1 μg/ml, or at a concentration of 1 μg/ml. In one embodiment, the actin binding protein is thymosyinβ4. In one embodiment, the actin binding protein is thymosyinβ4, which is present in the culture media at a concentration within a range of 0.1-10 μg/ml, 0.5-5 μg/ml, 0.75-2.5 μg/ml or 0.9-1.1 μg/ml. In one embodiment, the actin binding protein is thymosyinβ4, which is present in the culture media at a concentration of 1 μg/ml.

Agonists of the BMP (bone morphogenetic protein) pathway include agents, molecules, compounds, or substances capable of stimulating (activating or upregulating) the BMP signaling pathway, which biologically is activated by binding of BMP to a BMP receptor. BMP receptors (BMPRs) are activin receptor-like kinases (ALK) (e.g., type I BMP receptor, including but not limited to ALK2 and ALK3). In one embodiment, the BMP pathway agonist is selected from the group consisting of BMP4, BMP2, BMP6, BMP7, GDF6, and combinations thereof. In one embodiment, the BMP pathway agonist is present in the culture media at a concentration within a range of 5-50 ng/ml, 10-40 ng/ml, 15-30 ng/ml or 20-25 ng/ml or at a concentration of 20 ng/ml. In an embodiment, the BMP pathway agonist is BMP4. In an embodiment, the BMP pathway agonist is BMP4, which is present in the culture media at a concentration within a range of 5-50 ng/ml, 10-40 ng/ml, 15-30 ng/ml or 20-25 ng/ml. In an embodiment, the BMP pathway agonist is BMP4, which is present in the culture media at a concentration of 20 ng/ml.

Antagonists of the WNT pathway include agents, molecules, compounds, or substances capable of inhibiting (downregulating) the canonical Wnt/β-catenin signaling pathway, which biologically is activated by binding of a Wnt-protein ligand to a Frizzled family receptor. In one embodiment, the WNT pathway antagonist is selected from the group consisting of XAV939, ICG-001 (Foscenvivint), Capmatinib (INCB28060), endo-IWR-1, IWP-2, IWP-4, MSAB, CCT251545, KY02111, NCB-0846, FH535, LF3, WIKI4, Triptonide, KYA1797K, JW55, JW 67, JW74, Cardionogen 1, NLS-StAx-h, TAK715, PNU 74654, iCRT3, iCRT14, WIF-1, DKK1, Isoquercitrin, Lanatoside C, Gigantol, RCM-1, WIKI4, IQ-1, Adavivant, PRI-724, Tegatrabetan, and combinations thereof. In one embodiment, the WNT pathway antagonist is present in the culture media at a concentration within a range of 10-500 nM, 50-250 nM, 50-150 nM, 75-125 nM or at a concentration of 100 nM. In one embodiment, the WNT pathway antagonist is XAV939. In one embodiment, the WNT pathway antagonist is XAV939, which is present in the culture media at a concentration of 10-500 nM, 50-250 nM, 50-150 nM or 75-125 nM. In one embodiment, the WNT pathway antagonist is XAV939, which is present in the culture media at a concentration of 100 nM.

Agonists of the RA pathway include agents, molecules, compounds, or substances capable of stimulation of a retinoic acid receptor (RAR) that is activated by both all-trans retinoic acid and 9-cis retinoic acid. There are three RARs: RAR-alpha, RAR-beta and RAR-gamma, which are encoded by the RARA, RARB, RARG genes, respectively. Different retinoic acid analogs have been synthesized that can activate the retinoic acid pathway. Non-limiting examples of such compounds include TTNPB (agonist of RAR-alpha, beta and gamma), AM 580 (RARalpha agonist), CD 1530 (potent and selective RARgamma agonist), CD 2314 (selective RARbeta agonist), Ch 55 (potent RAR agonist), BMS 753 (RARalpha-selective agonist), Tazarotene (receptor-selective retinoid; binds RAR-beta and -gamma), Isotretinoin (endogenous agonist for retinoic acid receptors; inducer of neuronal differentiation), and AC 261066 (RARβ2 agonist). In some embodiments, the RA signaling pathway agonist is selected from the group consisting of: i) a retinoid compound, ii) a retinoid X receptor (RXR) agonist, and iii) a 25 retinoic acid receptor (RARs) agonist. In particular embodiments, the RA pathway agonist is selected from the group consisting of: retinoic acid, Sr11237, adapalene, EC23, 9-cis retinoic acid, 13-cis retinoic acid, 4-oxo retinoic acid, and All-trans Retinoic Acid (ATRA).

Accordingly, in one embodiment, the RA pathway agonist is selected from the group consisting of retinoic acid (RA), TTNPB, AM 580, CD 1530, CD 2314, CD 437, Ch 55, BMS 753, BMS 961, Tazarotene, Tamibarotene, Isotretinoin, Tretinoin, AC 261066, AC 55649, Sr11237, adapalene, EC23, 9-cis retinoic acid, 13-cis retinoic acid, 4-oxo retinoic acid, and All-trans Retinoic Acid (ATRA), AY 9944 dihydrochloride, Ciliobrevin A, Cyclopamine, or combinations thereof. In one embodiment, the RA pathway agonist is present in the culture media at a concentration within a range of 100-1000 nM, 200-800 nM, 250-750 nM or 400-600 nM, or at a concentration of 500 nM. In one embodiment, the RA pathway agonist is retinoic acid (RA). In one embodiment, the RA pathway agonist is retinoic acid (RA), which is present in the culture media at a concentration within a range of 100-1000 nM, 200-800 nM, 250-750 nM or 400-600 nM. In one embodiment, the RA pathway agonist is retinoic acid (RA), which is present in the culture media at a concentration of 500 nM.

Agonists of the WNT pathway include agents, molecules, compounds, or substances capable of stimulating (upregulating) the canonical Wnt/β-catenin signaling pathway, which biologically is activated by binding of a Wnt-protein ligand to a Frizzled family receptor. In one embodiment, a WNT pathway agonist is a glycogen synthase kinase 3 (Gsk3) inhibitor. In one embodiment, the WNT pathway agonist is selected from the group consisting of CHIR99021, CHIR98014, SB 216763, SB 415286, LY2090314, 3F8, A 1070722, AR-A 014418, BIO, BIO-acetoxime, AZD1080, WNT3A, alsterpaullone, indirubin-3-oxime, 1-azakenpaullone, kenpaullone, TC-G 24, TDZD 8, TWS 119, NP 031112, AT 7519, KY 19382, AZD2858, and combinations thereof. In one embodiment, the WNT pathway agonist is present in the culture media at a concentration within a range of 3.0-9.0 μM, 4.0-8.0 μM, 5.0-7.0 μM, 6.5-7.5 μM or at a concentration of 6.0 μM. In one embodiment, the WNT pathway agonist is CHIR99021. In one embodiment, the WNT pathway agonist is CHIR99021, which is present in the culture media at a concentration within a range of 3.0-9.0 μM, 4.0-8.0 μM, 5.0-7.0 μM or 6.5-7.5 μM. In one embodiment, the WNT pathway agonist is CHIR99021, which is present in the culture media at a concentration of 6.0 μM (e.g., in the stage 1 culture media for generating early mesoderm progenitors).

### III. Culture Conditions

In combination with the chemically-defined and optimized culture media described in subsection II above, the methods of generating hemogenic endothelial cells of the disclosure utilize standard culture conditions established in the art for cell culture. For example, cells can be cultured at 37° C. and under 5% CO₂ conditions. Cells can be cultured in standard culture vessels or plates, such as culture dishes, culture flasks or 96-well plates. Pluripotent stem cells can be cultured in commercially-available media prior to differentiation. For example, stem cells can be cultured for at least one day in in a specialized stem cell media such as Essential 8 Flex media (Thermo Fisher #A2858501) prior to the start of the differentiation protocol. In a non-limiting exemplary embodiment, stem cells are passaged onto vitronectin (Thermo Fisher #A14700) coated 6-well plates at 41,666, cells/cm 2 density and cultured for one day in Essential 8 Flex media prior to differentiation.

To begin the differentiation protocol starting from stem cells, the media the stem cells are being cultured in is changed to a basal differentiation media that has been supplemented with a Wnt signaling pathway agonists, such as a GSK-3β inhibitor (e.g., CHIR99021) as described above in subsection II, referred to herein as stage 1 of the differentiation protocol. A basal differentiation media can include, for example, a commercially-available base supplemented with additional standard culture media components needed to maintain cell viability and growth, but typically lacking serum (the basal differentiation media is a serum-free media).

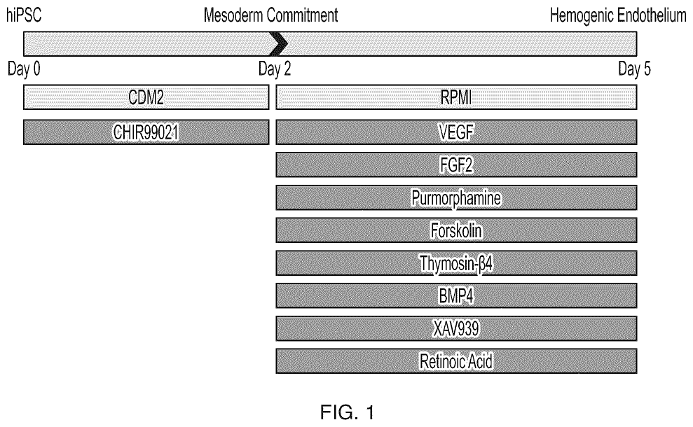

In a non-limiting exemplary embodiment, a basal differentiation media for stage 1 of the differentiation protocol is CDM2 media (as shown in FIG. 1) supplemented with 1% penicillin/streptomycin. The CDM2 media contains 0.5× IMDM (Thermo Fisher #12440046), 0.5× F12 (Thermo Fisher #11765047), poly(vinyl alcohol) (Sigma #p8136) at 1 mg/ml, chemically defined lipid concentrate (Thermo Fisher #11905031) at 1%, 1-thioglycerol (Sigma #M6145) at 450 μM, Insulin (Sigma #11376497001) at 0.7 μg/ml and transferrin (Sigma #10652202001) at 15 μg/ml.

In a non-limiting exemplary embodiment, a basal differentiation media for stage 2 of the differentiation protocol (as shown in FIG. 1) is commercially available RPMI media with 2% B-27 supplement, L-ascorbic acid at 100 μg/ml, and 1% penicillin/streptomycin.

In certain embodiments, the starting pluripotent stem cells are adhered to plates, preferably coated with an extracellular matrix material such as vitronectin. In one embodiment, the stem cells are cultured on a vitronectin coated culture surface (e.g., vitronectin coated 96-well plates).

The culture media typically is changed regularly to fresh media. For example, in one embodiment, media is changed every 24 hours.

To generate early mesoderm progenitors, the starting pluripotent stem cells are cultured in a media comprising a GSK-3β inhibitor (e.g., CHIR99021) for sufficient time for cellular differentiation and expression of committed early mesoderm progenitor-associated markers, typically two days. This protocol for generation of early mesoderm progenitors from PSCs is referred to herein as “step (a)” or “stage 1.”

To generate differentiated hemogenic endothelial cells from the early mesoderm progenitors, the progenitors are cultured in a media comprising a VEGFR agonist, an FGFR agonist, a sonic hedgehog (SHH) agonist, an adenylyl cyclase activator, an actin-binding protein, a BMP pathway agonist, a Wnt pathway antagonist, and a retinoic acid (RA) receptor agonist for sufficient time for cellular differentiation and expression of hemogenic endothelial cell-associated markers (e.g., CD31+CD34+CD143+CD309+GATA2+FLI1+RUNX1+vWF-F), typically three days. This protocol for generation of hemogenic endothelial cells from early mesoderm progenitors is referred to herein as “step (b)” or “stage 2.”

In various embodiments, the early mesoderm progenitors are cultured in the optimized culture media for sufficient time to increase the expression of at least one, and preferably a plurality of, hemogenic endothelial cell-associated markers. Non-limiting examples of suitable hemogenic EC-associated markers include CD31, CD34, CD143, CD309, GATA2, FLI1, RUNX1, and vWF. In embodiments, cells are cultured for sufficient time to increase the expression levels of at least two, at least three, at least four, at least five, at least six, at least seven or at least eight hemogenic EC-associated markers. In an embodiment, cells are cultured for sufficient time to increase the expression level of at least one hemogenic EC-associated marker by at least 10%, 20%, 30%, 40%, 50%, 60%, 70%, 80%, 90% or 100% as compared to the starting cell population. The level of expression of markers in the cultured HECs can be measured by techniques available in the art (e.g., RNAseq analysis and/or flow cytometry).

Accordingly, in the first stage of the method, which generates early mesoderm progenitors from PSCs, pluripotent stem cells are cultured in the stage 1-optimized culture media on days 0-2, or starting on day 0 and continuing through day 2, or for 48 hours (2 days), or for at least 36 hours, or at least 40 hours, or at least 44 hours, or at least 48 hours.

Accordingly, in the second stage of the method, which generates hemogenic endothelial cells from early mesoderm progenitors, the early mesoderm progenitors generated in stage 1 are replated and further cultured in the stage 2-optimized culture media on days 2-5, or starting on day 2 and continuing through day 5, or starting on day 2 and continuing for 72 hours (3 days), or starting on day 2 and continuing for at least 60 hours, or at least 64 hours, or at least 68 hours, or at least 70 hours, or at least 72 hours, or starting on day 2 and continuing for 60 hours, or for 64 hours, or for 68 hours, or for 70 hours or for 72 hours.

The culture media typically is changed regularly to fresh media. For example, in certain embodiments, media is changed every 24 hours, or every 48 hours or every 72 hours.

### IV. Uses

The methods and compositions of the disclosure for generating hemogenic endothelial cells allow for efficient and robust availability of these cell populations for a variety of uses. For example, the methods and compositions can be used in the study of hematopoietic development and differentiation, including biology to assist in the understanding of hematopoietic diseases and disorders. For example, the HECs generated using the methods of the disclosure can be further purified according to methods established in the art using agents that bind to surface markers expressed on the cells.

The HECs obtained according to the methods of the disclosure can be further cultured under blood lineage-specific culture conditions, leading to differentiation into short term and long term hematopoietic stem cells. Thus, the HECs obtained according to the methods of the disclosure offer the opportunity to investigate functional aspects of the hematopoietic system and its development. Other uses include for 3D-bioprinting, drug screening, safety assessments, vascular tissue engineering, and disease modeling.

The HECs generated according to the methods of the disclosure, or further differentiated hematopoietic-lineage cells derived therefrom, also are contemplated for use in the treatment of various hematopoietic diseases and disorders, for example through delivery of the cells to a subject having the disease or disorder. Examples of hematopoietic diseases and disorders include, but are not limited to, cancers such as leukemias and lymphomas, blood disorders, and autoimmune disorders.

### V. Compositions

In other aspects, the disclosure provides compositions related to the methods of generating hemogenic endothelial cells, including culture media and isolated cell cultures.

In one aspect, the disclosure provides a culture media for generating CD31+CD34+CD143+CD309+GATA2+FLI1+RUNX1+vWF+HECs comprising a VEGFR agonist, an FGFR agonist, a sonic hedgehog (SHH) agonist, an adenylyl cyclase activator, an actin-binding protein, a BMP pathway agonist, a Wnt pathway antagonist, and a retinoic acid (RA) receptor agonist. Non-limiting examples of suitable agents, and concentrations therefor, include those described in subsection II above. In one embodiment, the VEGFR agonist is VEGF, the FGFR agonist is FGF2, the SHH agonist is Purmorphamine, the adenylyl cyclase activator is Forskolin, the actin binding protein is thymosin-β4, the BMP pathway agonist is BMP4, the Wnt pathway antagonist is XAV939 and the RA receptor agonist is retinoic acid. In one embodiment, VEGF is at a concentration of 50 ng/ml, FGF2 is at a concentration of 10 ng/ml, Purmorphamine is at a concentration of 500 nM, Forskolin is at a concentration of 1 μM, thymosin-β4 is at a concentration of 1 μg/ml, BMP4 is at a concentration of 20 ng/ml, XAV939 is at a concentration of 100 nM and retinoic acid is at a concentration of 500 nM.

In another aspect, the disclosure provides an isolated cell culture of human hemogenic endothelial cells (ECs), the culture comprising: human CD31+CD34+CD143+CD309+GATA2+FLI1+RUNX1+vWF+HECs cultured in a culture media comprising a VEGFR agonist, an FGFR agonist, a sonic hedgehog (SHH) agonist, an adenylyl cyclase activator, an actin-binding protein, a BMP pathway agonist, a Wnt pathway antagonist, and a retinoic acid (RA) receptor agonist. Non-limiting examples of suitable agents, and concentrations therefor, include those described in subsection II above. In one embodiment, the VEGFR agonist is VEGF, the FGFR agonist is FGF2, the SHH agonist is Purmorphamine, the adenylyl cyclase activator is Forskolin, the actin binding protein is thymosin-β4, the BMP pathway agonist is BMP4, the Wnt pathway antagonist is XAV939 and the RA receptor agonist is retinoic acid. In one embodiment, VEGF is at a concentration of 50 ng/ml, FGF2 is at a concentration of 10 ng/ml, Purmorphamine is at a concentration of 500 nM, Forskolin is at a concentration of 1 μM, thymosin-β4 is at a concentration of 1 μg/ml, BMP4 is at a concentration of 20 ng/ml, XAV939 is at a concentration of 100 nM and retinoic acid is at a concentration of 500 nM.

The present invention is further illustrated by the following examples, which should not be construed as further limiting. The contents of figures and all references, patents and published patent applications cited throughout this application are expressly incorporated herein by reference.

### EXAMPLES

### Example 1: Culture Protocol Development for Generation of Hemogenic Endothelial Cells from Pluripotent Stem Cells

A two-stage recipe for generation of hemogenic endothelial cells was developed that can guide human IPSC to hemogenic endothelial cells expressing CD31, CD34, CD143, CD309, GATA2, FLI1, RUNX1, vWF, and VE-cadherin after 5 days in culture. HECs were generated from pluripotent stem cells using a two-stage protocol, illustrated schematically in FIG. 1.

In brief, starting PSCs were first differentiated into early mesoderm progenitors by culture of the PSCs in a media comprising a GSK-3β inhibitor, based on protocols established in the art for early differentiation along the endothelial lineage. In an embodiment, the PSCs are cultured in a media comprising CHIR99021 at 6 μM for two days (days 0-2).

The early mesoderm progenitors were used for further differentiation along the endothelial lineage using a High-Dimensional Design of Experiments (HD-DoE) approach to simultaneously test multiple process inputs (e.g., small molecule agonists or antagonists) on output responses, such as gene expression. Based on predicted conditions that maximize expression of hemogenic endothelial enriched genes such as CD31, CD34, CD143, CD309, GATA2, Fill and RUNX1, vWF, a complex recipe was developed for generating differentiated hemogenic endothelial cells from the early mesoderm progenitors, composed of 8 agents as shown below in Table 1:

TABLE 1
Hemogenic Endothelial Cell Generation Culture Media Recipe

| Agent | Role | Concentration | |
|---|---|---|---|
| VEGF | VEGFR agonist | 50 | ng/mL |
| FGF2 | FGFR agonist | 10 | ng/mL |
| Purmorphamine | SHH pathway agonist | 500 | nM |
| Forskolin | Adenylyl cyclase activator | 1 | μM |
| Thymosin-β4 | Actin binding protein | 1 | μg/ml |
| BMP4 | BMP pathway agonist | 20 | ng/ml |
| XAV939 | WNT pathway antagonist | 100 | nM |
| Retinoic acid (RA) | RA pathway agonist | 500 | nM |

This recipe is referred to herein as the stage 2 recipe for generating hemogenic endothelial cells.

The development of the two-stage recipe is described in detail below.

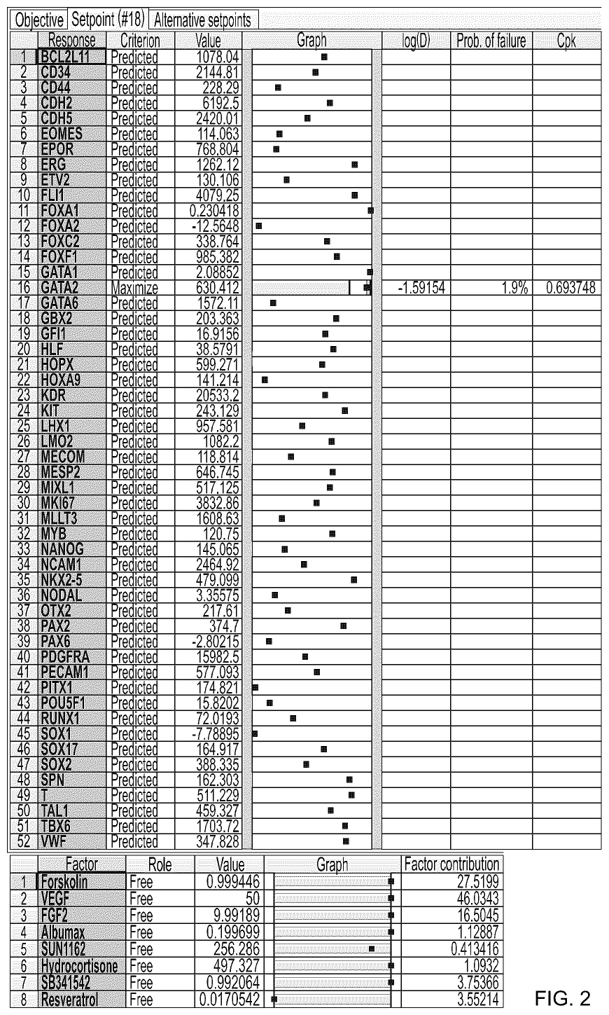

First, undifferentiated iPSC were grown for 48 h in CDM2 media containing 6 uM CHIR99021 (referred to herein as stage 1). Then stage 2 media was engineered. The basis of this was an 8-factor HD-DoE experiment with focus on differentiation of cells toward the hemogenic endothelial lineage for an additional 3 days after termination of stage 1 treatment. To test the effectors, 48 different combinations of effectors generated using Design-of-Experiments compression through D-optimality were robotically prepared. The effector combinations were prepared in a basal media and were subsequently added to the cells, which were then allowed to differentiate. Three days later, RNA extraction was performed, and gene expression was obtained using quantitative PCR analysis. The data were normalized and modeled using partial least squares regression analysis to the effector design, resulting in the generation of gene-specific models, which after model tuning for maximal Q2 predictive power, provided explanation of the effectors ability to control the expression of individual genes, combinatorically, and individually. Solutions within the tested space could then be explored to address desirability. For stage 2, conditions were created inducing the expression of GATA2, a transcription factor identified as a master regulator of hematopoiesis. GATA2 promotes the generation of HECs and facilitates endothelial-to-hematopoietic transition (EHT). GATA2 also suppresses cardiac differentiation at the mesoderm stage, directing cells to a hematopoietic fate. Optimizing for maximal expression of GATA2 led to a robust solution. At this solution, other genes were also predicted to be abundantly expressed, such as CD34, ERG FLI1, HOPX, KDR, LMO2, and CD31, all genes highly expressed on hemogenic endothelial cells, suggesting cell commitment to this lineage (FIG. 2). In this model, high levels of KDR, important receptor for VEGF signaling, were observed. Also, GATA6, PAX6, OTX2, and SOX1, were downregulated (gene associated with the ectoderm and endoderm). Additionally, genes related to the earlier stages such as, NODAL and EOMES, were downregulated, indicating that cells are differentiating. This model was derived from initial testing of eight factors including: Forskolin, VEGF, FGF2, Albumax, SUN1162, Hydrocortisone, SB341542, and Resveratrol. Three of these effectors: Forskolin, VEGF, and FGF2, showed positive impact on expression of genes of interest with 27, 46 and 17 factor contributions, respectively (FIG. 2). Factors with low contribution were excluded. Within the specifications of attaining approximate 86% maximal expression of GATA2, this complex media composition had a Cpk value (process capability index) of 0.69, with a corresponding 1.9% risk of failure.

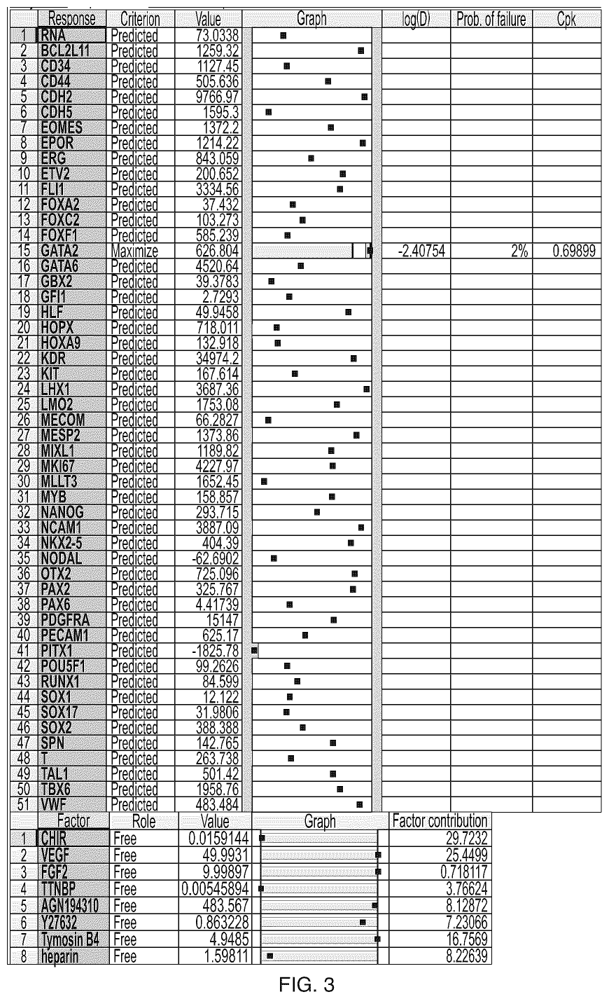

Next, further factors were evaluated to possibly increase the complexity of the signaling inputs to attain effective fate control. As previously, expression of GATA2 was focused on. Optimizing for maximal expression of the GATA2 led to a robust solution. At this solution, other genes were also predicted to be abundantly expressed, such as CD34, CD44, ERG, FLI1, KDR, LMO2, TAL1, and vWF, all genes related to the hemogenic endothelial program suggesting that cells are commitment to this lineage (FIG. 3). High levels of KDR were again observed. This model was derived from initial testing of eight factors including: CHIR, VEGF, FGF2, TTNBP, AGN194310, Y27632, Thymosin-β4, and heparin. Two of these effectors: VEGF and Thymosin-β4, showed positive impact on expression of genes of interest with high factor contribution of 25 and 16 factor contributions, respectively (FIG. 3). CHIR, TTNBP, AGN194310, Y27632, and heparin where not included in this recipe, as sufficient GATA2 induction was obtained without them and had a relatively low factor contribution. Although FGF2 had a low factor contribution in this model, it was included in the recipe as it had >16 factor contribution in 2 of 3 models. Within the specifications of attaining approximate 87% maximal expression of GATA2, this complex media composition had a Cpk value (process capability index) of 0.7, with a corresponding 2% risk of failure.

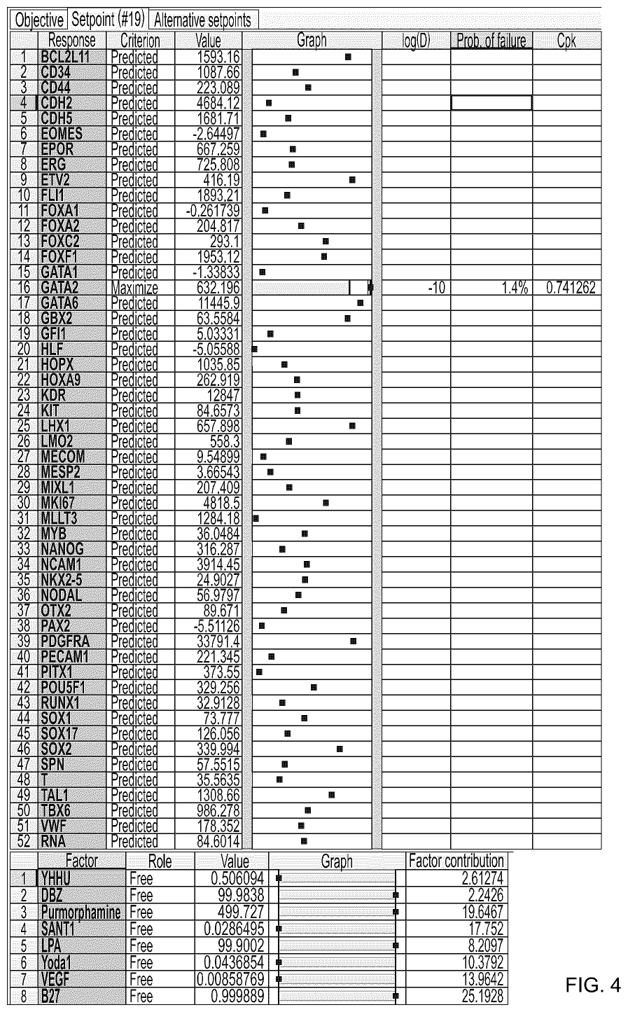

To further improve the recipe for hemogenic endothelial cell differentiation, additional HD-DoE experiments were performed. This model was derived from initial testing of eight factors including: YHHU, DBZ, Purmorphamine, SANT1, LPA, Yodal, VEGF, and B-27. Two of these effectors: Purmorphamine and B-27 showed positive impact on expression of genes of interest with 20 and 25 factor contributions, respectively (FIG. 4). Other than VEGF, all remaining factors were not included in this recipe, as sufficient GATA2 induction was obtained without them and had a relatively low factor contribution. VEGF remained in the recipe as 4 out of 5 models portrayed a strong positive effect towards GATA2 induction. In this solution, other genes were also predicted to be abundantly expressed, such as CD34, ERG, FLI1, ETV2, and TAL1, all genes highly expressed on hemogenic endothelial cells, suggesting cell commitment to this lineage (FIG. 4). Within the specifications of attaining approximate 84% maximal expression of GATA2, this complex media composition had a Cpk value (process capability index) of 0.7, with a corresponding 1.4% risk of failure.

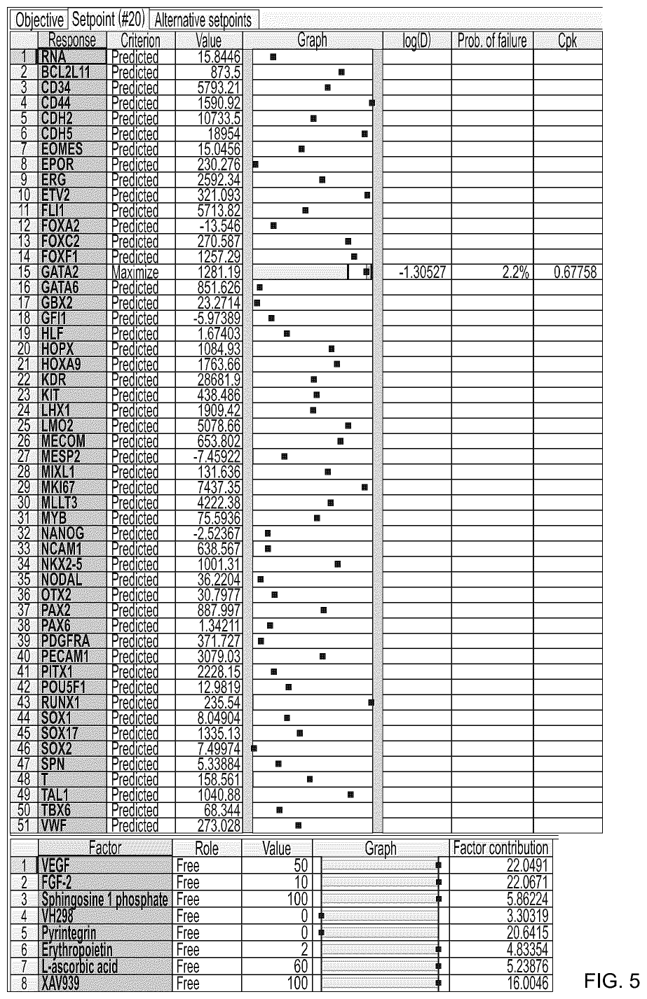

A fourth modelling experiment was conducted to continue improving the recipe for hemogenic endothelial cell differentiation. This model was derived from initial testing of eight factors including: VEGF, FGF2, Sphingosine 1 phosphate, V11298, Pyrintegrin, Erythropoietin, L-ascorbic acid, and XAV939. Four of these effectors: VEGF, FGF2, L-ascorbic acid, and XAV939 showed positive impact on expression of genes of interest with 22, 22, 5, and 16 factor contributions, respectively (FIG. 5). L-ascorbic acid was included as it is in the basal media. All remaining factors were not included in this recipe, as sufficient GATA2 induction was obtained without them and had a relatively low factor contribution. In this solution, other genes were also predicted to be abundantly expressed, such as CD34, CD44, CDH5, LMO2, MECOM, and TAL1, all genes highly expressed on hemogenic endothelial cells, suggesting cell commitment to this lineage (FIG. 5). Within the specifications of attaining approximate 82% maximal expression of GATA2, this complex media composition had a Cpk value (process capability index) of 0.7, with a corresponding 2.2% risk of failure.

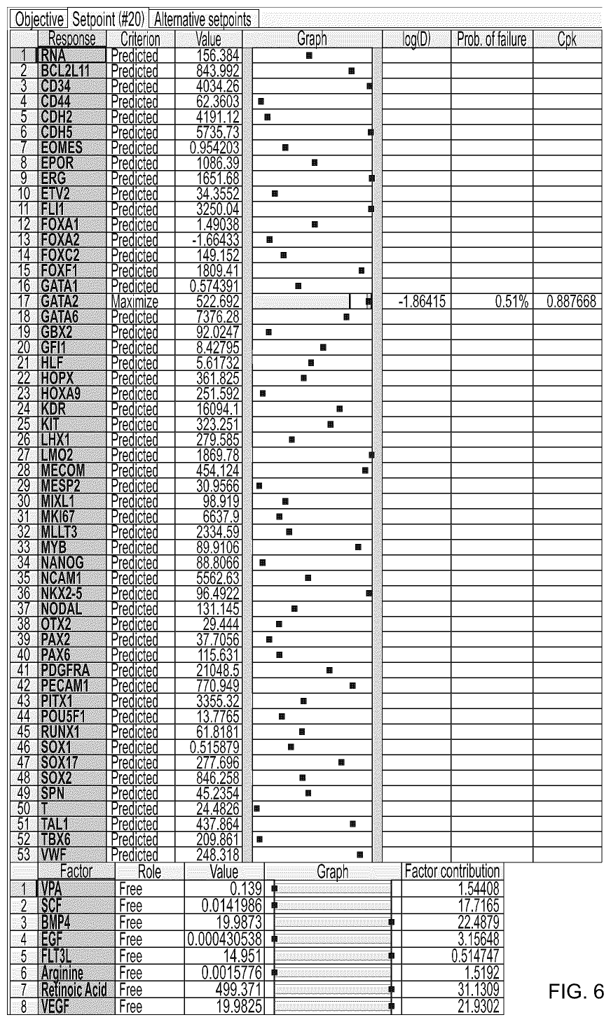

A fifth and final model was conducted to finalize the optimization of the recipe for hemogenic endothelial cell differentiation. This model was derived from initial testing of eight factors including: VPA, SCF, BMP4, EFG, FLT3L, Arginine, Retinoic Acid, and VEGF. Three of these effectors: Retinoic Acid, BMP4, and VEGF showed positive impact on expression of genes of interest with 22, 31, and 22 factor contributions, respectively (FIG. 6). All remaining factors were not included in this recipe, as sufficient GATA2 induction was obtained without them and had a relatively low factor contribution. In this solution, other genes were also predicted to be abundantly expressed, such as CD34, CDH5, ERG, FLI1, LMO2, and CD31, all genes highly expressed on hemogenic endothelial cells, suggesting cell commitment to this lineage (FIG. 6). Within the specifications of attaining approximate 81% maximal expression of GATA2, this complex media composition had a Cpk value (process capability index) of 0.9, with a corresponding 0.51% risk of failure. Considering all the models analyzed, based on predicted conditions that maximize expression of hemogenic endothelial enriched genes such as GATA2, LMO2, ERG, CDH5, CD31, FLI1, CD34, TAL1, MECOM, MYB, a complex recipe for hemogenic endothelial differentiation was developed that is composed of 8 effectors, as shown above in Table 1.

### Example 2: Factor Criticality Analysis of Hemogenic Endothelial Cell-Inducing Culture Conditions

The various contribution factors for each protocol input for the stage 2 culture recipe inducing hemogenic endothelial cells suggested variable relative influence. To assess the impact of elimination of each validated factor on genes associated with the endothelial lineage, dynamic profile analysis was used and the expression levels of genes of interest were compared in the absence of each finalized factor while others are kept present. Since expression levels of genes of interest reveal whether the desired outcome is reachable, this factor criticality analysis revealed the extent of importance of each input effector.

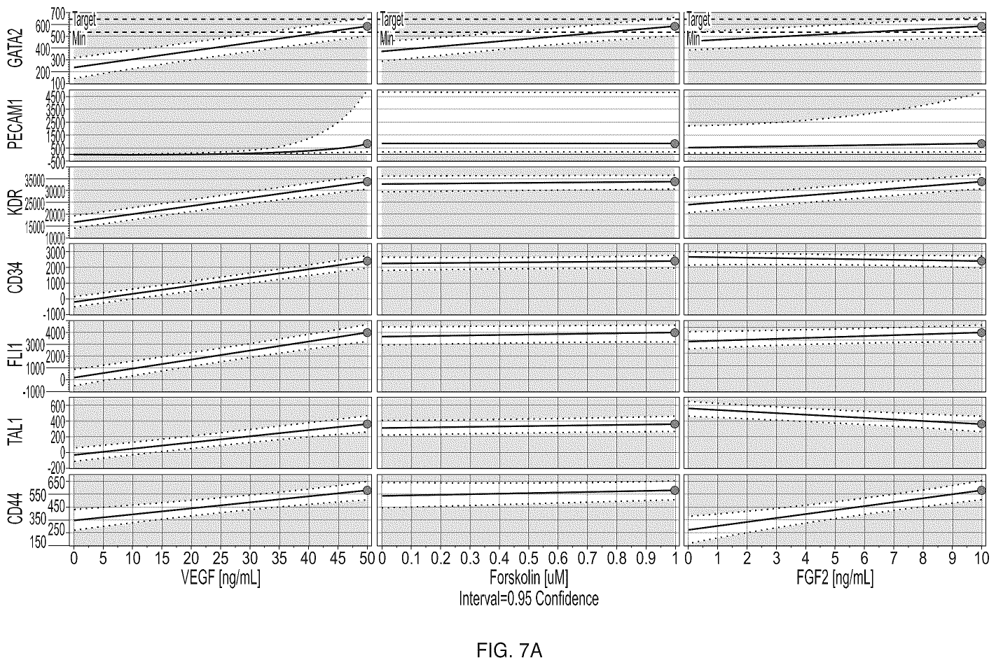

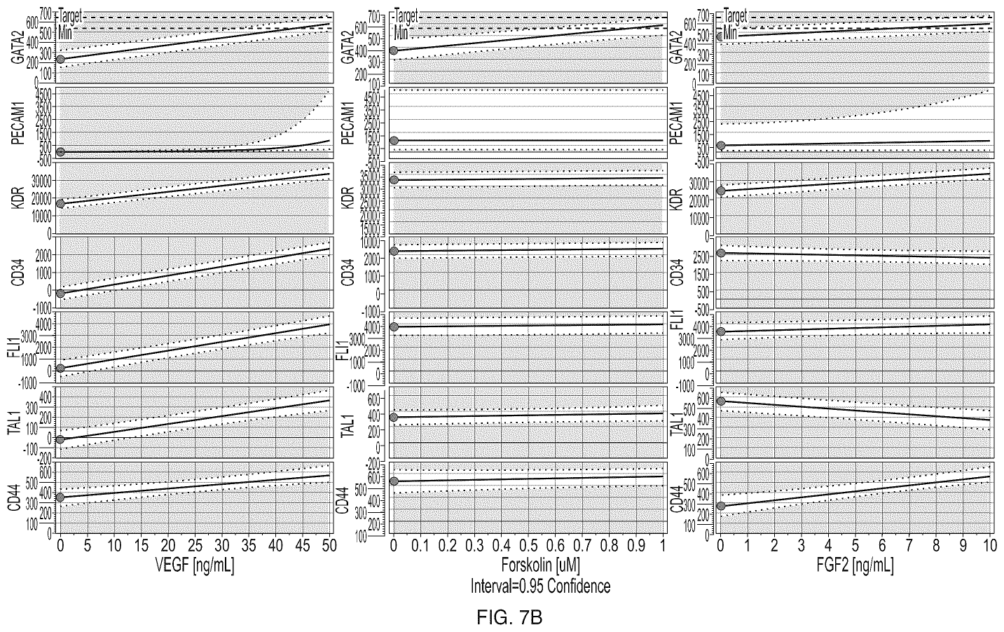

In this analysis, removal of a finalized effector while keeping other factors was used to analyze the impact of factors on the expression levels of GATA2, CD31, KDR, CD34, FLI1, TAL1, and CD44 (FIG. 7A). When VEGF was removed, values of GATA2 decreased from 583 to 236, values of CD31 changed from 879 to 2, values of KDR decreased from 33751 to 16856, values of CD34 decreased from 2350 to 150, values of Fill decreased from 3971 to 241, values of TAL1 decreased from 370 to 18, and values of CD44 decreased from 578 to 349. All changes represent a significant loss of expression of a desired gene (FIG. 7B). When Forskolin was removed, values of GATA2 decreased from 583 to 373, values of KDR decreased from 33751 to 32839, values of CD34 decreased from 2350 to 2240, values of Fill decreased from 3971 to 3740, values of TAL1 decreased from 370 to 318, and values of CD44 decreased from 578 to 540 (FIG. 7B). GATA2 had a significant decrease in expression when Forskolin was removed. When FGF2 was removed, values of GATA2 decreased from 583 to 458, values of CD31 decreased from 879 to 505, values of KDR decreased from 33760 to 23868, values of Fill decreased from 3970 to 3374, and values of CD44 decreased from 576 to 274 (FIG. 7B). This data shows that FGF2 is crucial for KDR, CD31, and CD44 expression.

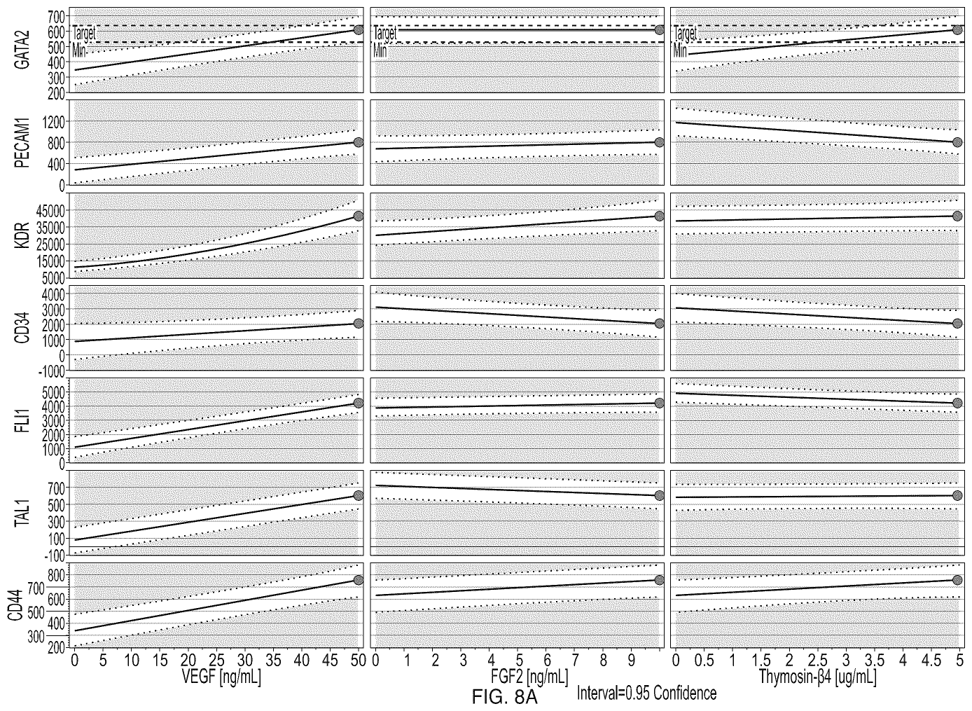

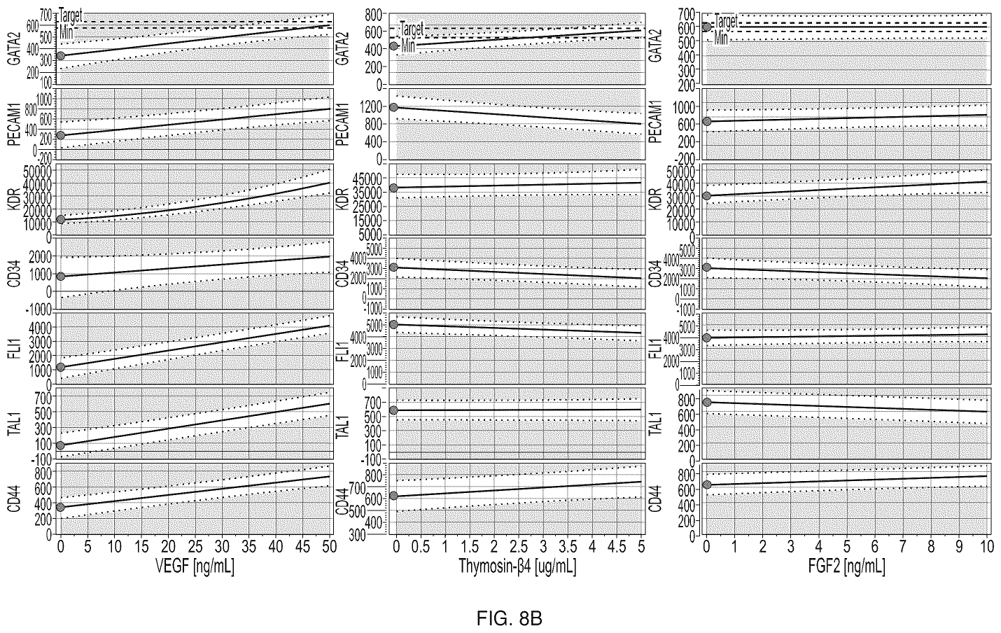

In another model, the effect of VEGF, FGF2, and Thymosin-β4 on the expression levels of GATA2, CD31, KDR, CD34, FLI1, TAL1, and CD44 was evaluated (FIG. 8A). Again, VEGF was critical for increasing the expression of all hemogenic endothelial genes analyzed. When FGF2 was removed, values of CD31 decreased from 807 to 677, values of KDR decreased from 40630 to 29902, and values from CD44 decreased from 748 to 626 (FIG. 8B). There was a significant loss of expression in KDR in the absence of FGF2. When Thymosin-β4 was removed, values of GATA2 decreased from 633 to 437, values of KDR decreased from 40660 to 37788, and values from CD44 decreased from 748 to 626 (FIG. 8B). Thymosin-β4 addition is required for GATA2 upregulation, therefore it was used in the stage 2 recipe.

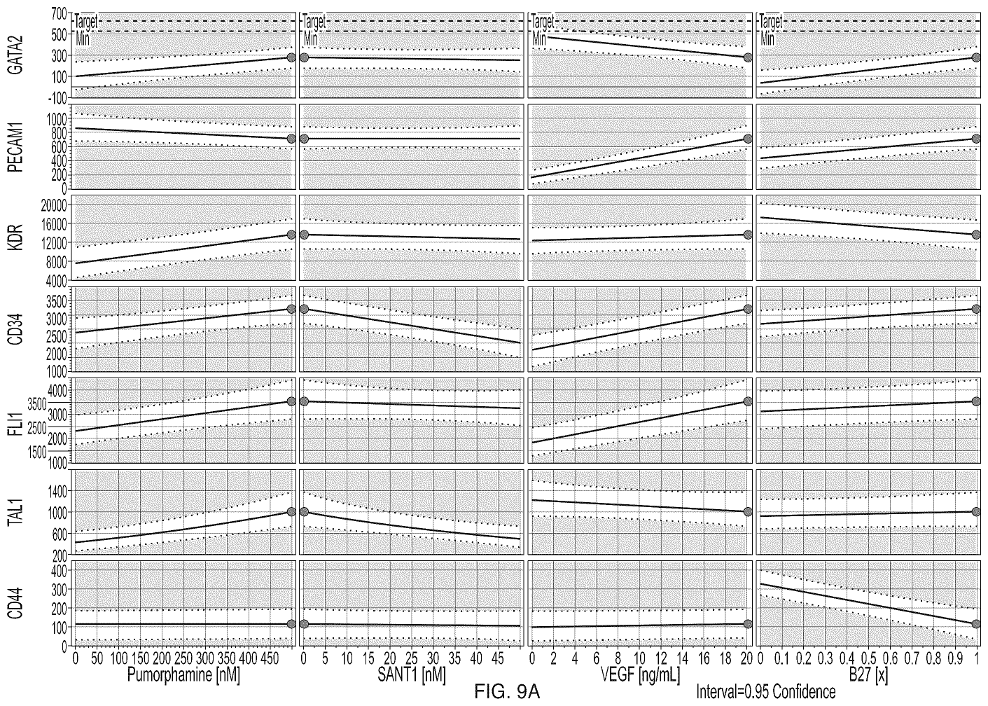

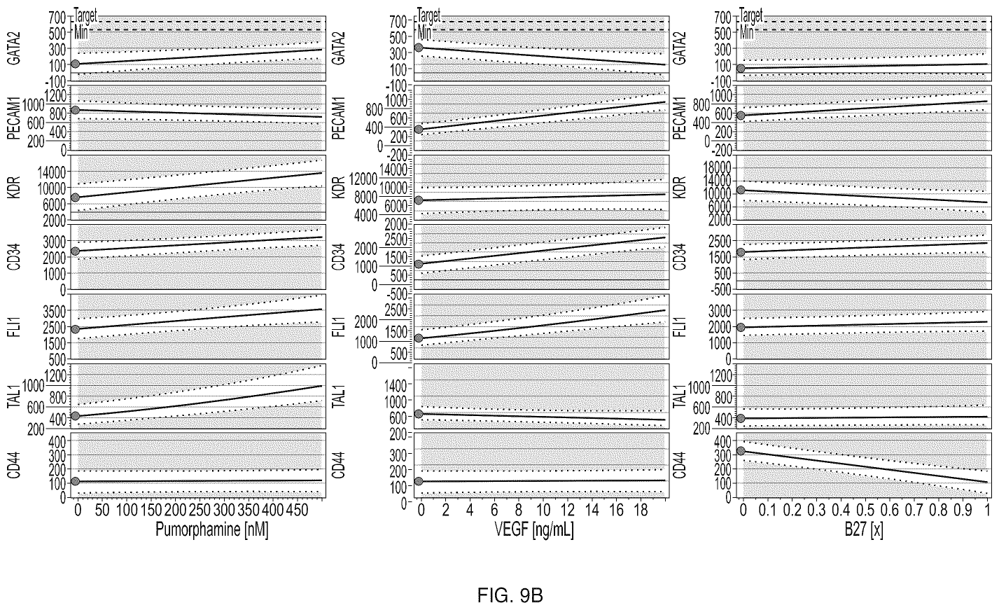

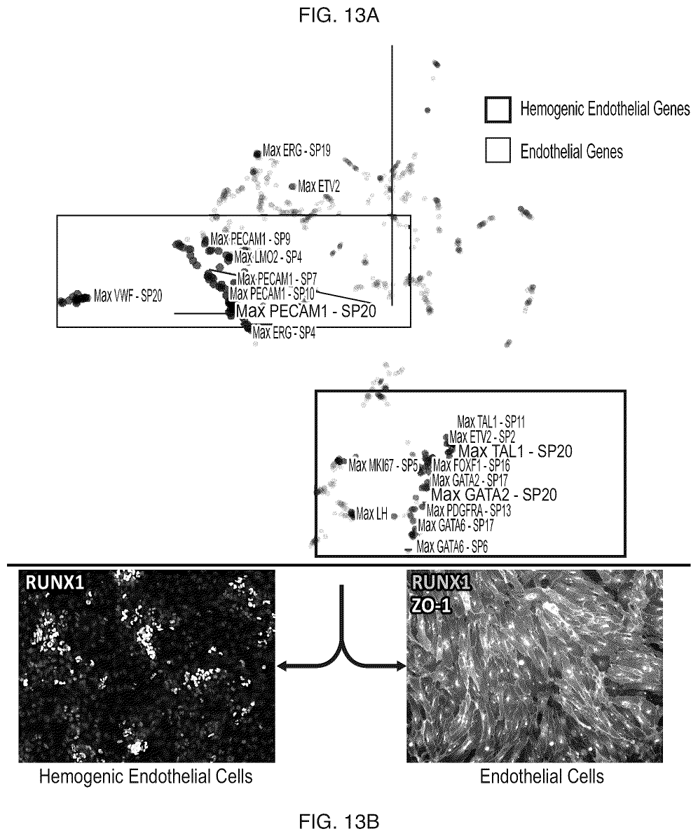

In another model, the effect of purmorphamine, a sonic hedgehog (SHH) activator, on the expression levels of GATA2, CD31, KDR, CD34, FLI1, TAL1, and CD44 (FIG. 9A) was evaluated. When doing this dynamic profile analysis, B-27 was kept in the model, since it is present in the basal media for validation experiments. When purmorphamine was removed, GATA2 levels decreased from 275 to 105, KDR levels decreased from 13612 to 7614, CD34 levels decreased from 3230 to 2369, Fill levels decreased from 3571 to 2324, TAL1 levels decreased from 1008 to 428. There was a significant loss in expression of GATA2, KDR, FLI1, and TAL1 in the absence of purmorphamine (FIG. 9B). When VEGF was removed, CD31 levels decreased from 869 to 262, KDR levels decreased to 7518 to 6209, CD34 levels decreased from 2341 to 868, and Fill levels decreased from 2292 to 974. There was a significant loss of expression in CD31, KDR, CD34, and Fill in the absence of VEGF (FIG. 9B). This model reveals that B-27 plays a synergistic role with the factors, contributing to increased gene expression in all but CD44 and KDR (FIG. 9A-9B). This model portrayed that the SHH pathway is a critical regulator of the hemogenic endothelial program. The addition of SANT1, a SHH antagonist, decreased levels of all genes of interest, most significantly: KDR from 13469 to 12504, CD34 from 3159 to 1995, and TAL1 from 1010 to 498 (FIG. 9A). This model indicates that the SHH pathway is a regulator of endothelial cell phenotype by acting as a lineage determinant pathway. The data shows that the SHH pathway is a critical regulator of endothelial differentiation, as the inhibition of the SHH pathway with SANT1 drives the cells to an endothelial lineage and activation of the pathway with purmorphamine drives the cells to a hemogenic endothelial lineage (FIG. 13A). Because RUNX1 is enriched on hemogenic endothelial cells, we compared RUNX1 expression on cells cultured with our hemogenic endothelial recipe versus cells cultured with an endothelial recipe containing SANT1. FIG. 13B shows RUNX1 expression just on hemogenic endothelial cells, not on endothelial cells. The activation of the SHH pathway is critical for the differentiation of HECs, thus purmorphamine was included in the stage 2 recipe.

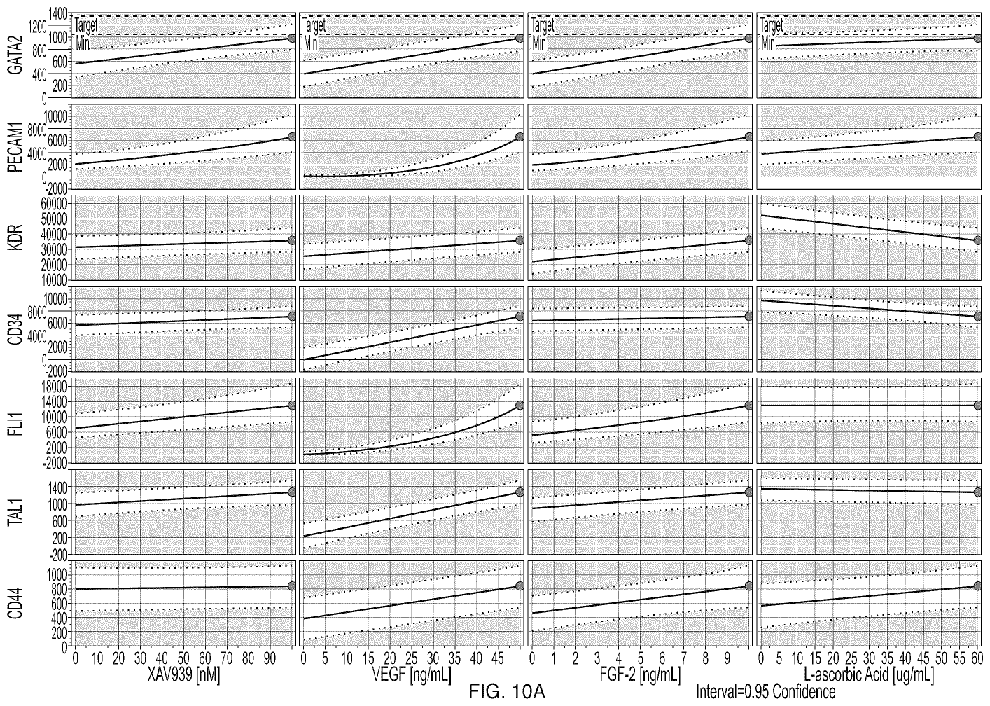

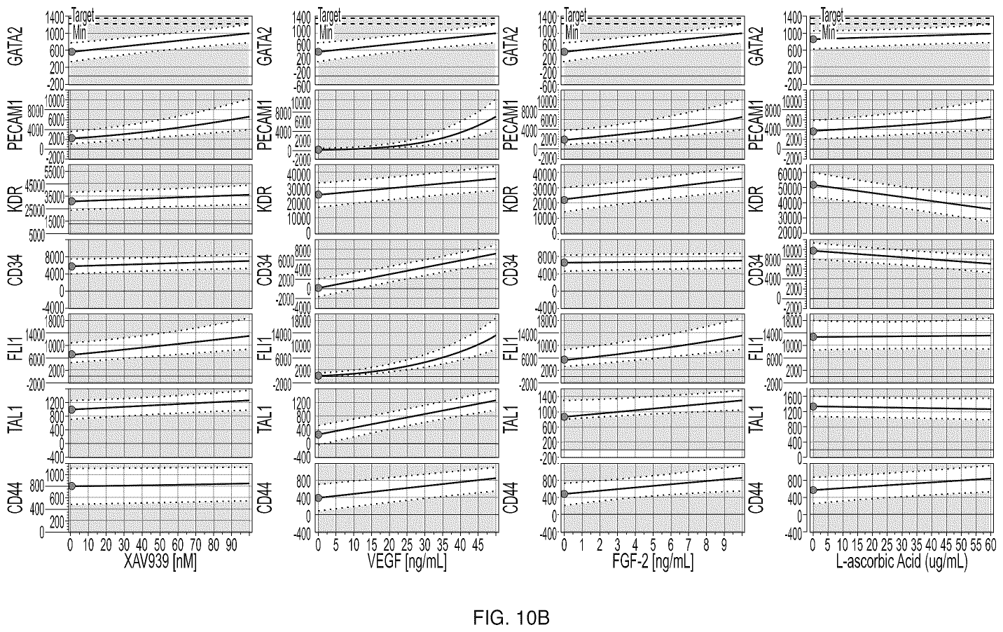

In another model, the effect of XAV939 and FGF2 on the expression levels of GATA2, CD31, KDR, CD34, FLI1, TAL1, and CD44 was evaluated (FIG. 10A). When doing this dynamic profile analysis, L-ascorbic acid was kept in the model, since it is present in the basal media and increased expression of crucial genes (FIG. 10B). Again, VEGF was critical for expression of all hemogenic endothelial genes analyzed. When XAV939 was removed, GATA2 levels decreased from 992 to 560, CD31 levels decreased from 6619 to 2181, KDR levels decreased from 36067 to 31179, CD34 levels decreased from 7050 to 5740, Fill levels decreased from 13094 to 7237, TAL1 levels decreased from 1265 to 982, and CD44 decreased from 838 to 799. There was a significant loss in expression of GATA2, CD31, CD34, and Fill in the absence of XAV939 (FIG. 10B). When FGF2 was removed GATA2 levels decreased from 992 to 397, CD31 levels decreased from 6619 to 2002, KDR levels decreased from 36067 to 22308, CD34 levels decreased from 7050 to 6550, Fill levels decreased from 13094 to 5406, TAL1 levels decreased from 1265 to 866, and CD44 decreased from 838 to 466 (FIG. 10B). There was a significant loss in expression of GATA2, CD31, KDR, FLI1, and CD44 with the absence of FGF2 (FIG. 10B). This model, along with the previous models, reveals that FGF2 plays a robust synergistic role with the factors, contributing to increased gene expression and thus selected for the stage 2 recipe.

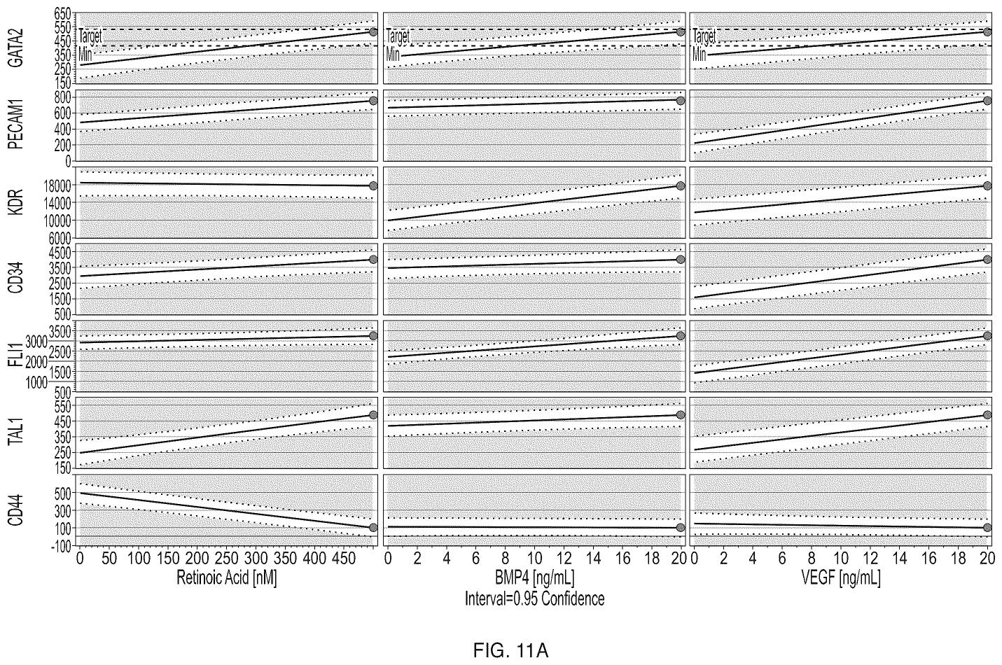

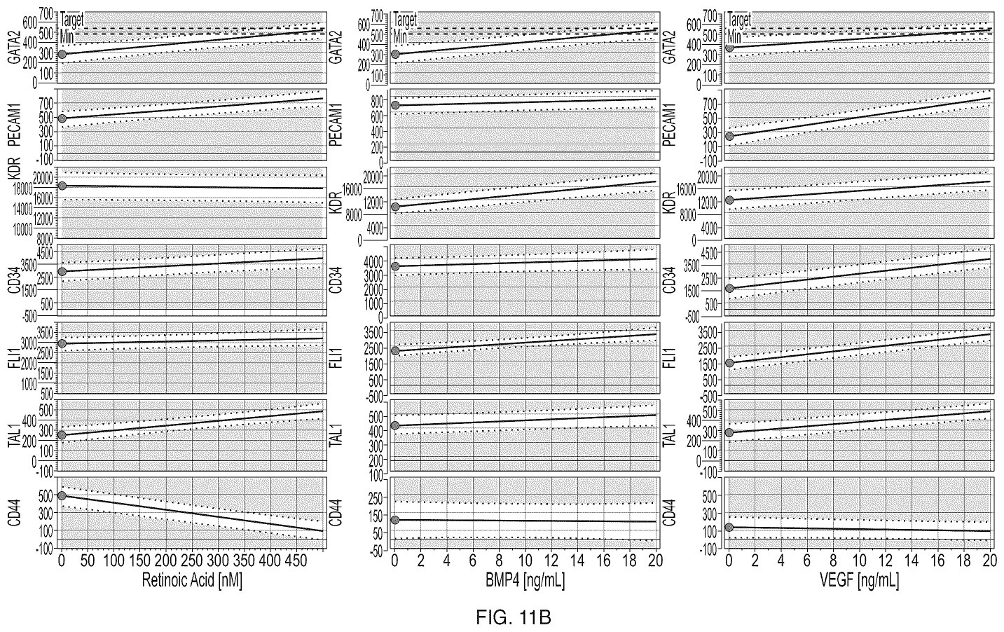

In the final model, the effect of retinoic acid and BMP4 on the expression levels of GATA2, CD31, KDR, CD34, FLI1, TAL1, and CD44 was evaluated (FIG. 11A). Again, VEGF was critical for expression of all hemogenic endothelial genes analyzed. When retinoic acid was removed GATA2 levels decreased from 519 to 281, CD31 levels decreased from 768 to 486, CD34 levels decreased from 3971 to 2902, Fill levels decreased from 3250 to 2936, and TAL1 levels decreased from 490 to 235 (FIG. 11B). There was a significant loss in expression of GATA2, CD31, and TAL1 in the absence of retinoic acid. When BMP4 was removed GATA2 levels decreased from 519 to 338, CD31 levels decreased from 768 to 682, KDR levels decreased from 17667 to 10025, CD34 levels decreased from 3971 to 3455, Fill levels decreased from 3250 to 2213, and TAL1 levels decreased from 490 to 422 (FIG. 11B). There was a significant loss in expression of GATA2, KDR, FLI1, and CD44 with the absence of FGF2 (FIG. 10B).

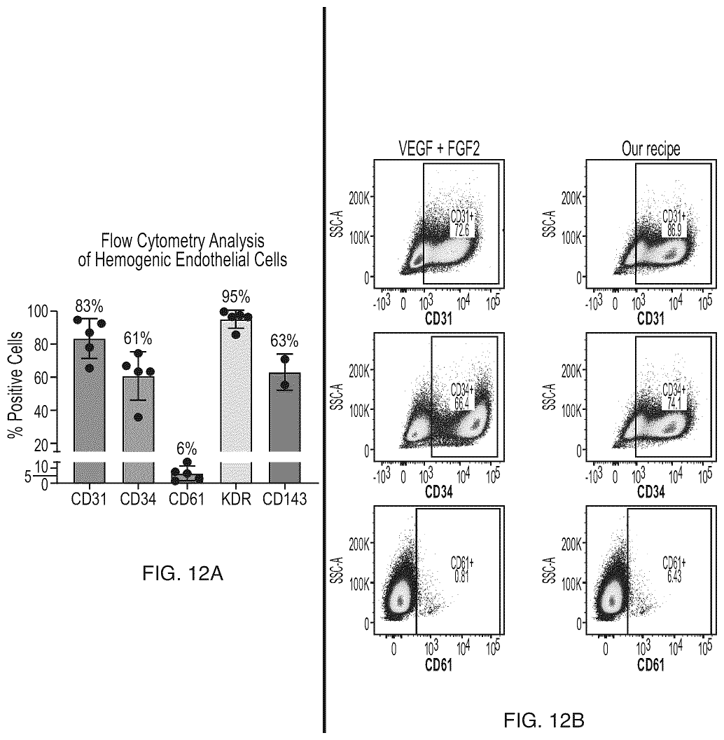

For additional supporting evidence of the stage 2 recipe, an experiment was conducted in the presence and absence of effectors in the stage 2 recipe. In this experiment, VEGF2 and FGF2 (two common factors used for endothelial differentiation) only, were compared against the complete stage 2 recipe. FIG. 12B illustrates the increase of CD31, CD34, and CD61 expression with the addition of the small molecules. CD61 has been shown to be a marker for HECs (Huang, K. et al. (2016) Stem Cell Reports 7:854-868) find is significantly upregulated when the stage 2 effectors were added to the recipe. The combination of the stage 2 effectors with VEGF and FGF guide the cells to differentiate into HECs. The complete composition of this stage 2 recipe can be found in Table 1.

### Example 3: Flow Cytometry Analysis and Immunocytochemistry of Stem Cell Derived Hemogenic Endothelial Cells Expressing Hemogenic Endothelial Cell Markers

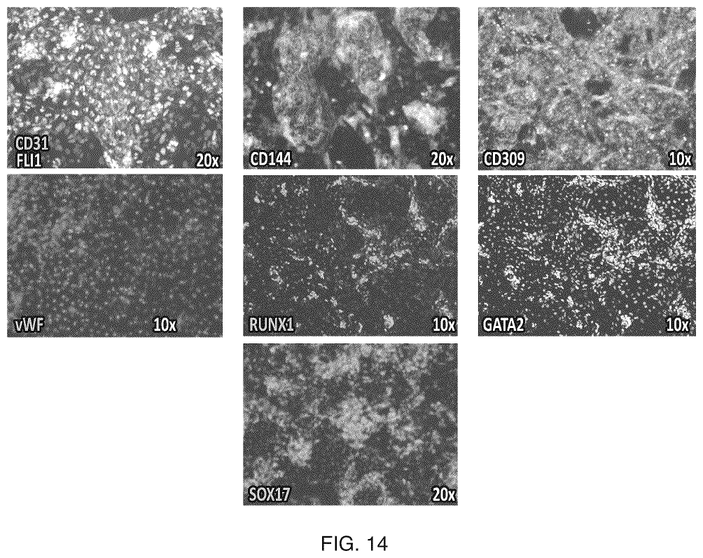

To further validate the developed recipe in Example 1, iPSCs were grown for 2 days in stage 1 media, and then cells were replated and grown for 3 days in stage 2 media, and flow cytometry and immunohistochemistry analysis was used to evaluate expression of hemogenic endothelial cell markers. The basal differentiation media used for this experiment is RPMI media with 2% B-27 supplement, L-ascorbic acid at 100 μg/ml, and 1% penicillin/streptomycin. Flow cytometry analysis confirmed the efficiency of the stage 2 recipe to promote conversion of iPSC to hemogenic endothelial cells (FIG. 12A-12B). 83% of cells were positive for CD31, 61% of cells were positive for CD34, 6% of cells were positive for CD61, 95% of cells were positive for CD309 (KDR) (FIG. 12A). 63% of cells were positive for CD143 (FIG. 12A). Additionally, immunofluorescence staining showed robust staining for various hemogenic endothelial markers such as CD31, FLI1, RUNX1, GATA2, vWF, and CD309 (KDR), and SOX17 (FIG. 14).

### Example 4: Flow Cytometry Analysis of Stem Cell Derived Hemogenic Endothelial Cells Expressing Hemogenic Endothelial Cell Markers from 2 separate iPSC Cell Lines

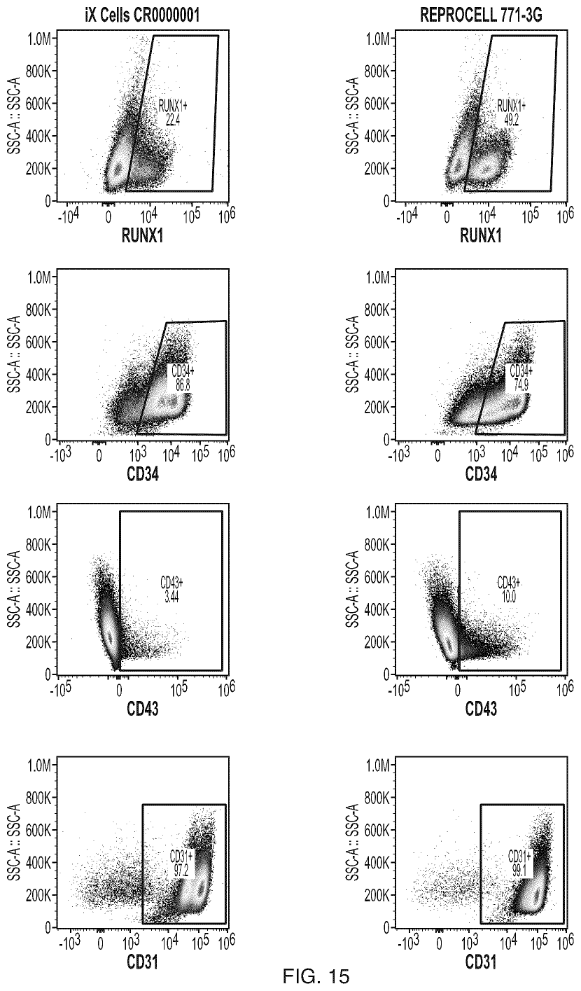

To further validate the developed recipe described in Example 1 in another cell line, iPSCs from 2 cell lines (iX Cells CR0000001 and REPROCELL 771-3G) were grown for 2 days in stage 1 media, and then cells were replated and grown for 3 days in stage 2 media, and flow cytometry analysis was used to evaluate expression of hemogenic endothelial cell markers. The basal differentiation media used for this experiment was RPMI media with 2% B-27 supplement, L-ascorbic acid at 100 μg/ml, and 1% penicillin/streptomycin. Flow cytometry analysis confirmed the efficiency of the stage 2 recipe to promote conversion of iPSC to hemogenic endothelial cells in both iPSC cell lines. FIG. 15 shows 22% vs 49% of cells expressing RUNX1, 87% vs 75% of cells expressing CD34, 3% vs 10% of cells expressing CD43, and 97% vs 99% of cells expressing CD31 in iX Cells CR0000001 vs REPROCELL 771-3G respectively.

### Example 5: RNA-Seq Analysis to Characterize iPSC-Derived Hemogenic Endothelial Cells

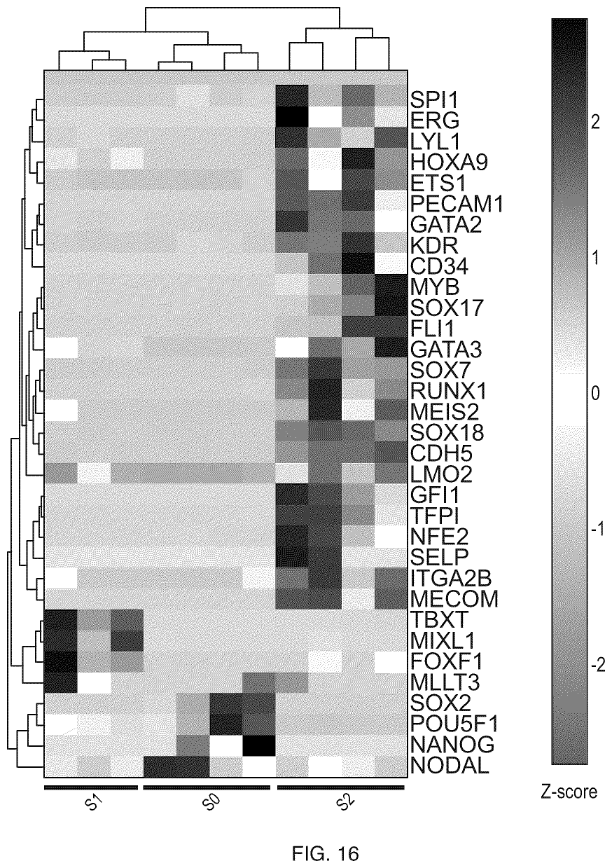

Bulk RNA-seq analysis was used to characterize iPSC-derived hemogenic endothelial cells (FIG. 16). iPSCs were differentiated to lateral plate mesoderm as previously described. Then, mesodermal cells were treated with stage 2 media for 3 days. As shown in FIG. 16, S0 cells express pluripotency markers such as NANOG, SOX2 and POUF51. S1 cells express genes associated with primitive streak and mesoderm such TBXT, MIXL1 and FOXF1. Finally, bulk RNAseq validation confirmed expression of blood and endothelial transcripts in the S2 cells. Transcription factors related to blood that were expressed were: RUNX1, GATA2, GATA3, FLI1, GFI1, MEIS2, MECOM and SPI1. For endothelial transcripts that were expressed included: SOX17, SOX7, SOX18, KDR, PECAM1, ERG, ETS1 and CDH5.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A method of generating human CD31+CD34+CD143+CD309+GATA2+FLI1+RUNX1+vWF+HECs) comprising:
culturing human early mesoderm progenitor cells in a culture media comprising a VEGFR agonist, an FGFR agonist, a sonic hedgehog (SHH) agonist, an adenylyl cyclase activator, an actin-binding protein, a BMP pathway agonist, a Wnt pathway antagonist, and a retinoic acid (RA) receptor agonist to generate human CD31+CD34+CD143+CD309+GATA2+FLI1+RUNX1+vWF+HECs.

2. The method of claim 1, wherein the early mesoderm progenitor cells are cultured for three days in the culture media.

3. The method of claim 1, wherein the early mesoderm progenitor cells are obtained by culturing human pluripotent stem cells in a media comprising a Wnt pathway agonist for two days.

4. A method of generating human CD31+CD34+CD143+CD309+GATA2+FLI1+RUNX1+vWF+HECs comprising:
(a) culturing human pluripotent stem cells in a culture media comprising a Wnt pathway agonist on day 0-day 2 to generate early mesoderm progenitor cells; and
(b) culturing the early mesoderm progenitor cells in a culture media comprising a VEGFR agonist, an FGFR agonist, a sonic hedgehog (SHH) agonist, an adenylyl cyclase activator, an actin-binding protein, a BMP pathway agonist, a Wnt pathway antagonist, and a retinoic acid (RA) receptor agonist on day 2-day 5 to generate human CD31+CD34+CD143+CD309+GATA2+FLI1+RUNX1+vWF hemogenic (ECs).

5. The method of claim 3, wherein the Wnt pathway agonist is a GSK-3β inhibitor.

6. The method of claim 5, wherein the GSK-3β inhibitor is CHIR99021.

7. The method of claim 6, wherein CHIR99021 is present in the culture at a concentration in a range of 3.0-9.0 μM.

8. The method of claim 7, wherein CHIR99021 is present in the culture at a concentration of 6.0 μM.

9. The method of claim 1, wherein the VEGFR agonist is VEGF.

10. The method of claim 9, wherein VEGF is present in the culture at a concentration in a range of 25-75 ng/ml.

11. The method of claim 9, wherein VEGF is present in the culture at a concentration of 50 ng/ml.

12. The method of claim 1, wherein the FGFR agonist is FGF2 or SUN11602.

13. The method of claim 12, wherein the FGFR agonist is present in the culture at a concentration in a range of 1-20 ng/ml.

14. The method of claim 12, wherein the FGFR agonist is FGF2, which is present in the culture at a concentration in a range of 1-20 ng/ml.

15. The method of claim 12, wherein the FGFR agonist is FGF2, which is present in the culture at a concentration of 10 ng/ml.

16. The method of claim 1, wherein the sonic hedgehog (SHH) agonist is selected from the group consisting of Purmorphamine, SSH, GSA 10, SAG, and combinations thereof.

17. The method of claim 16, wherein the SHH agonist is present in the culture at a concentration in a range of 100-1000 nM.

18. The method of claim 16, wherein the SHH agonist is Purmorphamine, which is present in the culture at a concentration in a range of 400-600 nM.

19. The method of claim 16, wherein the SHH agonist is Purmorphamine, which is present in the culture at a concentration of 500 nM.

20. The method of claim 1, wherein the adenylyl cyclase activator is selected from the group consisting of Forskolin, NKH 477, PACAP 1-27, PACAP 1-38, Adenosine, Carbacyclin, Dopamine, Endothelin 1, Endothelin 1, L-(−)-Epinephrine-(+)-bitartrate, Glucagon, Isoproterenol HCl, (±)-Octopamine HCl, Parathyroid Hormone 1-34, Prostaglandin D₂, Prostaglandin E₁, Prostaglandin E₂, Prostaglandin I₂, [Arg⁸]-Vasopressin, [Lys⁸]-Vasopressin, and combinations thereof.

21. The method of claim 20, wherein the adenylyl cyclase activator is present in the culture at a concentration in a range of 0.1-10 μM.

22. The method of claim 20, wherein the adenylyl cyclase activator is Forskolin, which is present in the culture at a concentration in a range of 0.75-2.5 μM.

23. The method of claim 20, wherein the adenylyl cyclase activator is Forskolin, which is present in the culture at a concentration of 1.0 μM.

24. The method of claim 1, wherein the actin binding protein is selected from the group consisting of thymosyin-β4, HMRef, α-actinin, β-spectrin, dystrophin, utrophin, fimbrin, and combinations thereof.

25. The method of claim 24, wherein the actin binding protein is present in the culture at a concentration in a range of 0.1-10 μg/ml.

26. The method of claim 24, wherein the actin binding protein is thymosyin-β4, which is present in the culture at a concentration in a range of 0.75-2.5 μg/ml.

27. The method of claim 24, wherein the actin binding protein is thymosyin-β4, which is present in the culture at a concentration of 1 μg/ml.

28. The method of claim 1, wherein the BMP pathway agonist is selected from the group consisting of BMP4, BMP2, BMP6, BMP7, GDF6, and combinations thereof.

29. The method of claim 28, wherein the BMP pathway agonist is present in the culture at a concentration in a range of 5-50 ng/ml.

30. The method of claim 28, wherein the BMP pathway agonist is BMP4, which is present in the culture at a concentration in a range of 15-30 ng/ml.

31. The method of claim 28, wherein the BMP pathway agonist is BMP4, which is present in the culture at a concentration of 20 ng/ml.

32. The method of claim 1, wherein the Wnt pathway antagonist is selected from the group consisting of XAV939, ICG-001 (Foscenvivint), Capmatinib (INCB28060), endo-IWR-1, IWP-2, IWP-4, MSAB, CCT251545, KY02111, NCB-0846, FH535, LF3, WIKI4, Triptonide, KYA1797K, JW55, JW 67, JW74, Cardionogen 1, NLS-StAx-h, TAK715, PNU 74654, iCRT3, iCRT14, WIF-1, DKK1, Isoquercitrin, Lanatoside C, Gigantol, RCM-1, WIKI4, IQ-1, Adavivant, PRI-724, Tegatrabetan, or combinations thereof.

33. The method of claim 32, wherein the Wnt pathway antagonist is present in the culture at a concentration in a range of 10-500 nM.

34. The method of claim 32, wherein the Wnt pathway antagonist is XAV939, which is present in the culture at a concentration in a range of 50-150 nM.

35. The method of claim 32, wherein the Wnt pathway antagonist is XAV939, which is present in the culture at a concentration of 100 nM.

36. The method of claim 1, wherein the retinoic acid (RA) pathway agonist is selected from the group consisting of retinoic acid (RA), TTNPB, AM 580, CD 1530, CD 2314, CD 437, Ch 55, BMS 753, BMS 961, Tazarotene, Tamibarotene, Isotretinoin, Tretinoin, AC 261066, AC 55649, Sr11237, adapalene, EC23, 9-cis retinoic acid, 13-cis retinoic acid, 4-oxo retinoic acid, and All-trans Retinoic Acid (ATRA), AY 9944 dihydrochloride, Ciliobrevin A, Cyclopamine, or combinations thereof.

37. The method of claim 36, wherein the RA pathway agonist is present in the culture at a concentration in a range of 100-1000 nM.

38. The method of claim 36, wherein the RA pathway agonist is retinoic acid, which is present in the culture at a concentration in a range of 400-600 nM.

39. The method of claim 36, wherein the RA pathway agonist is retinoic acid, which is present in the culture at a concentration of 500 nM.

40. The method of claim 3, wherein the pluripotent stem cells are embryonic stem cells.

41. The method of claim 3, wherein the pluripotent stem cells are induced pluripotent stem cells.

42. A culture media for generating HECs comprising a VEGFR agonist, an FGFR agonist, a sonic hedgehog (SHH) agonist, an adenylyl cyclase activator, an actin-binding protein, a BMP pathway agonist, a Wnt pathway antagonist, and a retinoic acid (RA) receptor agonist.

43. The culture media of claim 42, wherein the VEGFR agonist is VEGF, the FGFR agonist is FGF2, the SHH agonist is Purmorphamine, the adenylyl cyclase activator is Forskolin, the actin binding protein is thymosin-β4, the BMP pathway agonist is BMP4, the Wnt pathway antagonist is XAV939 and the RA receptor agonist is retinoic acid.

44. The culture media of claim 43, wherein VEGF is at a concentration of 50 ng/ml, FGF2 is at a concentration of 10 ng/ml, Purmorphamine is at a concentration of 500 nM, Forskolin is at a concentration of 1 μM, thymosin-β4 is at a concentration of 1 μg/ml, BMP4 is at a concentration of 20 ng/ml, XAV939 is at a concentration of 100 nM and retinoic acid is at a concentration of 500 nM.

45. An isolated cell culture of human hemogenic endothelial cells, the culture comprising: human CD31+CD34+CD143+CD309+GATA2+FLI1+RUNX1+vWF+HECs cultured in a culture media comprising a VEGFR agonist, an FGFR agonist, a sonic hedgehog (SHH) agonist, an adenylyl cyclase activator, an actin-binding protein, a BMP pathway agonist, a Wnt pathway antagonist, and a retinoic acid (RA) receptor agonist.
